(19) United States
(12) Patent Application Publication
(10) Pub. No.: US 2004/0133355 A1
(43) Pub. Date: Jul. 8, 2004

(54) Methods and compositions utilizing evolutionary computation techniques and differential data sets

(75) Inventor: Luke V. Schneider, Palo Alto, CA (US)

Correspondence Address: Robin M. Silva, Esq., Dorsey & Whitney LLP, Intellectual Property Department, Four Embarcadero Center, Suite 3400, San Francisco, CA, 94111-4187, US

(73) Assignee: Target Discovery, Inc.

(21) Appl. No.: 10/678,041

(22) Filed: Oct. 1, 2003

Related U.S. Application Data
(60) Provisional application No. 60/415,481, filed on Oct. 1, 2002.

(51) Int. Cl. G06F 19/00; G01N 33/48; G01N 33/50

(52) U.S. Cl. 702/19; 702/20

(57) ABSTRACT

Systems biology models of biochemical systems have proved to be powerful conceptual tools for the analysis of biological data but have historically been arduous to formulate and test. Biological data is also unique in that it exists mostly in the differential display format, unsuitable for use in standard mathematical modeling efforts. The present invention removes the drudgery of model building and data analysis from the shoulders of the individual researchers—who are no longer able assimilate the overwhelming volume of bioinformatic data and synthesize this into a model of the underlying physiology—by providing an artificial intelligence (Al) substitute. In addition to the use of Al methods for building the invention describes the use of difference equations and linear algebra to recast the models into another mathematical domain, allowing the direct use of differential display data formats for model testing and eliminating the need for time-consuming numerical integration. The combined effect is to significantly accelerate the model building and testing process and provide more complete alternative models for physiological and other complex systems.

This application claims benefit of priority under 35 U.S.C. §119(e) to U.S. S. No. 60/415,481, hereby expressly incorporated by reference in its entirety.

### BACKGROUND OF THE INVENTION

The completion of the human genome is expected to dramatically expand drug discovery horizons. It has been estimated that 5,000 or more new drug discovery targets will emerge over the next several years from the thousands of genes that have been newly uncovered. A decade ago, fewer than 10% of human genes were known, which necessarily severely restricted drug discovery possibilities. In fact, it has been estimated that all of the prescription drugs now available attack a mere 500 targets.

In reality, the human genome has been revealed to be much smaller than was widely anticipated: 30,000 to 40,000 genes versus earlier estimates of 100,000 or more. However, this does not imply that drug discovery possibilities are much more limited than was previously believed. It is apparent that much of the complexity of human biology lay not in simple gene number, but rather in gene product number. One gene, as it turns out, makes more than one protein product, contrary to old biological dogma. As such, 30,000 genes could well translate into 250,000 or more distinct proteins, many of which may constitute viable drug discovery targets. In addition, protein-protein interactions constitute another potential set of drug discovery targets. The total potential number of new drug discovery targets is not limited to gene number, but actually should be some multiple of that figure. This large number of potential proteins are organized into complex and multidimensional metabolic and signaling pathways to effect a biological goal.

Signaling pathways in cells often begin with an effector stimulus that leads to a phenotypically describable change in cellular physiology. Despite the key role intracellular signaling pathways play in disease pathogenesis, in most cases, little is understood about a signaling pathway other than the initial stimulus and the ultimate cellular response. Metabolic pathways are typically highly branched structures, evolving to allow the synthesis or catabolism of large numbers of metabolites needed to supply the building blocks and energy for the cell to survive in widely varying situations. These metabolic networks are constructed much like electric grids, such that if one pathway is blocked others can supply the necessary molecules through round about means.

Historically, signal transduction and metabolic pathways have been analyzed by biochemistry or genetics. The biochemical approach dissects a pathway in a “stepping-stone” fashion: find a molecule that acts at, or is involved in, one end of the pathway, isolate assayable quantities and then try to determine the next molecule in the pathway, either upstream or downstream of the isolated one. The genetic approach is classically a “shot in the dark”: induce or derive mutants in a signaling pathway and map the locus by genetic crosses or complement the mutation with a cDNA library. Limitations of biochemical approaches include a reliance on a significant amount of pre-existing knowledge about the constituents under study and the need to carry such studies out in vitro, post-mortem. Limitations of purely genetic approaches include the need to first derive and then characterize the pathway before proceeding with identifying and cloning the gene.

Traditionally, biochemical signaling pathways have been elucidated slowly, sequentially, and with significant “wet chemistry” research time. While the metabolic pathways are sometimes known in whole or in part from the literature, mathematical models of these pathways are usually unavailable. With the advent of high throughput mRNA, protein, and metabolite profiling methods, it is now possible to generate concentration and flux data to which models can be fit. These biological models can in turn be used to make useful predictions either of additional experiments to validate the model, the need for as yet unidentified biological components, or for determining the best means of intervention to alleviate symptoms, prevent consequences, or affect a cure.

Lederberg and McCray report 44 different “omics” technologies based on a search of MEDLINE conducted in 2001 (Lederberg et al., The Scientist 15:8 (2001). As Anderson and Sielhammer (Anderson et al., Electrophoresis 18:533 (1997) and Gygi et al. (Mol. Cell Biol. 19:1720 (1999) have aptly showed mRNA and protein levels do not correlate; therefore, evaluating a single “omic” dimension is problematic (e.g., mRNA expression in microarray data). Each instrumental analysis technique provides just one aspect of the biochemical changes occurring within the cell in response to disease or therapy. Drug discovery and development is rapidly becoming a variable-rich environment; there exists an ever-increasing amount of “omics” data:

Genomics, which includes:

Chemical Genomics, the analysis of genetic variability and its impact on efficacy and toxicology.

Expressional Genomics, the analysis of patterns of gene expression by tissue and disease state.

Proteomics, which includes:

Expressional Proteomics, the quantification and identification of every protein in a cell as a function of disease state.

Interactional Proteomics, the identification and quantification of every protein-protein and protein-ligand interaction

Structural Proteomics, the determination (empirically or by ab initio modeling) of the 3° structure of all proteins

Metomics, the quantitative determination of the small molecule fluxes in tissues, including:

Adsorption, metabolism, deposition, and excretion (ADME) of drugs.

Metabolomics, the quantification of anabolism, catabolism, and transport of metabolites in tissues and cells.

Cellomics, which includes:

Subcellular distribution, the identification of the subcellular localization of molecules of interest

Cytometry, the determination of cell type or properties (including pathology) by imaging or flow methods.

Two challenges in developing physiological or systems biology models are the complexity of the models themselves and the incompleteness, breadth, and quality of all the “omics” data that must be explained by the model. Incompleteness of data often allows more than one model to fit. The breadth of “omics” data available leads to parameter rich and complex models that are difficult to construct from first principles or human analysis of the problem. The quality of current “omics” data is often low (i.e., large standard deviations) or is over-specified with more than one set of low quality data being available for parameter determination. Finally, most “omics” data exists as ratiometric or relative abundance measurements, which do not supply the necessary absolute concentration data typically necessary to develop biochemical models. These types of data are often called “differential display”. Examples include: isotope coded affinity tags (ICAT™)¹ and isotope differentiated binding energy shift tags (IDBEST™) for protein expression analysis, 2 and stable isotope ratio methods for the comparison of relative metabolite ratios between two samples by nuclear magnetic resonance (NMR)³ and mass spectrometry (MS).⁴ 1 Aebersold, R. H., et al., WO 00/11208 (Mar. 2, 2000). 2 Schneider, L. V. et al., WO 01/49951 (Aug. 29, 2002); Hall, M. P. et al., paper presented at the Amer. Soc. Mass Spectro. 50^{th} Annual Meeting, Orlando, Fla. (June, 2002). 3 Gonzalez, B., Piotto, M., and G. Huber, WO 02/31523A1 (Apr. 18, 2002). 4 Schneider, L. V. et al., WO 00/63683 (Oct. 26, 2000).

However, recent work has focused on the use of computational techniques, similar to those used to model circuits in the electrical engineering field, to allow for the analysis of systems biology. Systems biology, further described below, is the mathematical modeling of biochemical pathways by integrating the data produced from the growing number of biological databases (e.g. genomics, proteomics, metomics, cellomics data, etc., referred to herein as “OMICS” data), as well as experimental data, to produce knowledge about the underlying biochemistry of a disease or cellular state.

A variety of methods have been described for the analysis of different types of data using evolutionary computational techniques, see U.S. Pat. No. 6,282,527 B1; U.S. Pat. Nos. 5,914,891; 6,051,029; 6,078,739; 6,069,629; 5,947,899; WO 99/27443; WO 00/63793; WO 00/65523; WO 01/98935; WO 02/05205; WO 02/44992, all of which are incorporated by reference in their entirety.

However, there exists a significant need for methods that allow for the development and evolution of systems biology models where information about the underlying natural biochemistry is not fully known or is incomplete. In addition, methods useful in building models useful in biological settings may potentially also be used in other complicated and variable-rich “systems”, such as traffic patterns, weather patterns, financial and market analyses, etc. A specific additional need is to develop modeling approaches that can fit differential display data formats.

Accordingly, the present invention provides compositions and methods useful to generate, elucidate and complete mathematical models of complicated systems for which individual steps follow mathematical equations. Additionally, the present invention provides a means to cast models as difference equations so that model parameters can be fit directly to differential display “omics” data types. Finally, the present invention provides a means to cast the difference equation models in other mathematical domains to speed their solution.

### SUMMARY OF THE INVENTION

In accordance with the objects outlined herein, the present invention provides methods comprising providing a plurality of unit operations that represent all or a subset of all actions that can be done on a set of system components. The methods comprise providing a first hypothetical mathematical model comprising a subset of the unit operations and applying a first artificial intelligence (Al) algorithm to the first hypothetical mathematical model to produce a second hypothetical mathematical model. In one embodiment, a fitness function is used to filter the second hypothetical model to generate at least a third hypothetical mathematical model. Generally the fitness function is based on empirical data. Alternatively, the second model is compared directly with empirical data to define differences between the data and the model. These methods can be iterated as desired.

In an additional aspect, the methods to generate a mathematical model of a biological system comprising providing a plurality of first order pseudogene unit operations that represent all or a subset of all actions that can be done on a set of biochemical system components. The method comprises generating a first set of first order pseudochromosomes from the pseudogenes and applying a genetic algorithm with a fitness function to the set of first order pseudochromosomes to produce a second set of second order pseudochromosomes, with optional reiteration.

In a further aspect, the methods comprise methods of adjusting the algorithm used. In this embodiment, the methods comprise providing a plurality of unit operations that represent all or a subset of all actions that can be done on system components, and applying a first artificial intelligence (Al) algorithm to the plurality to produce a second hypothetical mathematical model. The second hypothetical model is compared to at least a first set of empirical data to define at least a first difference between the first hypothetical model and the data and altering the first algorithm to adjust for the first difference to generate a second Al algorithm. The second Al algorithm is applied to the second hypothetical model to produce a third hypothetical model which is compared to the first set of data.

In an additional aspect, the invention provides a computer readable memory to direct a computer to function in a specified manner, comprising a unit operations module to receive and store unit operations and generate at least a first hypothetical mathematical model, an analysis module to apply an artificial intelligence algorithm and a comparison module to compare hypothetical models to at least a first set of empirical data.

### DETAILED DESCRIPTION OF THE DRAWINGS

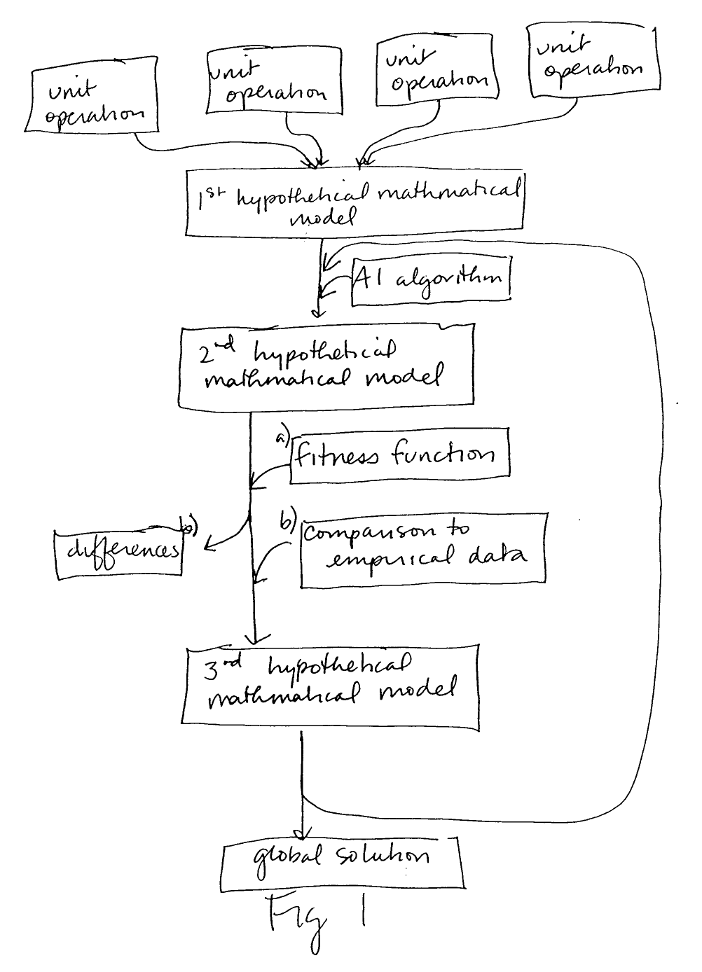

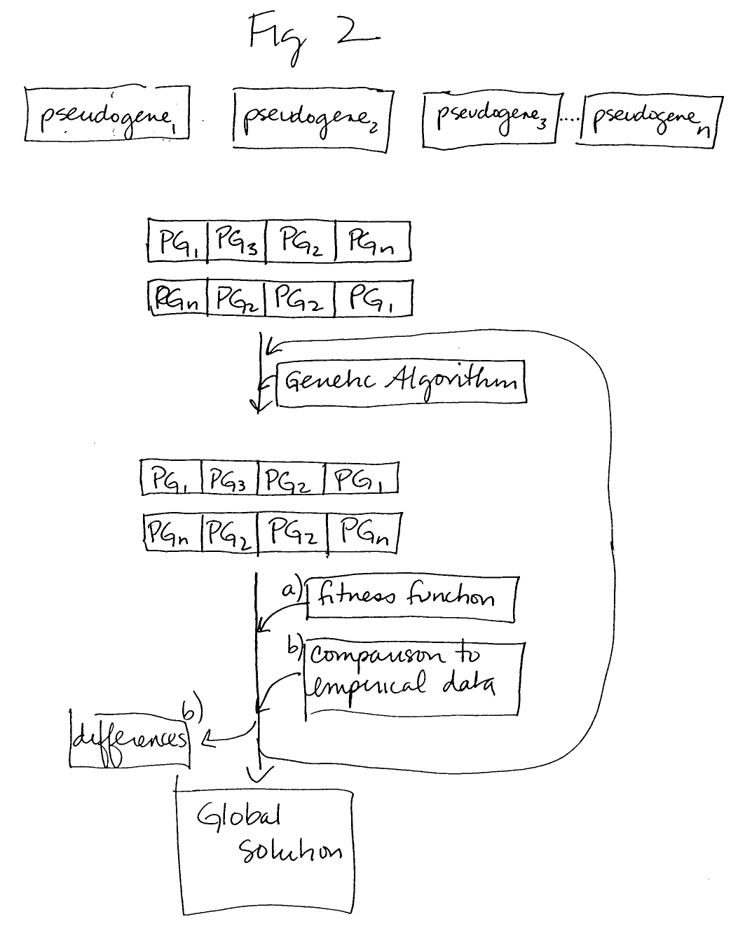

FIG. 1 depicts a schematic of the general method of the invention. Unit operations are used to create a first hypothetical mathematical model, upon which an Al algorithm is executed, to create a second mathematical model. Generally, a fitness function is applied (a)) which in some cases is a direct comparison to empirical data, to generate a third hypothetical mathematical model. This can then be iterated to find a global solution (although as outlined herein, convergence to a global solution is not required).

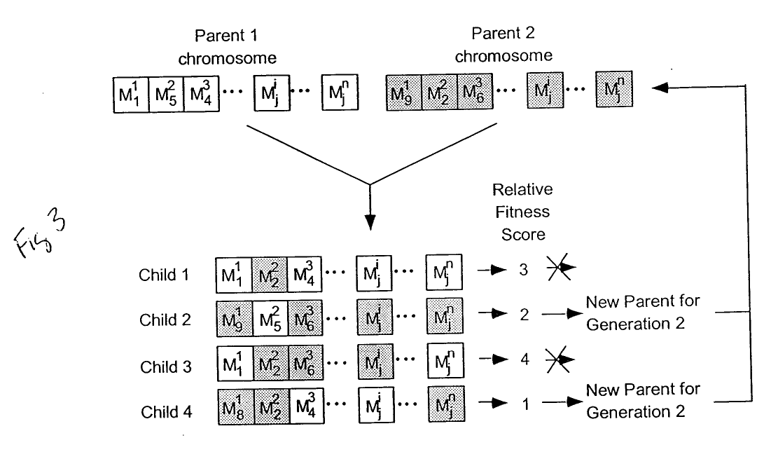

FIG. 2 depicts a schematic of the general GA method of the invention. Unit operations in the form of pseudogenes are used to create a first hypothetical mathematical model (e.g. in this case, two parent pseudochromosomes), upon which a genetic algorithm is executed, to create a second mathematical model (e.g. first generation child pseudochromosomes). Generally, a fitness function is applied (a)) which in some cases is a direct comparison to empirical data. This can then be iterated to find a global solution (although as outlined herein, convergence to a global solution is not required).

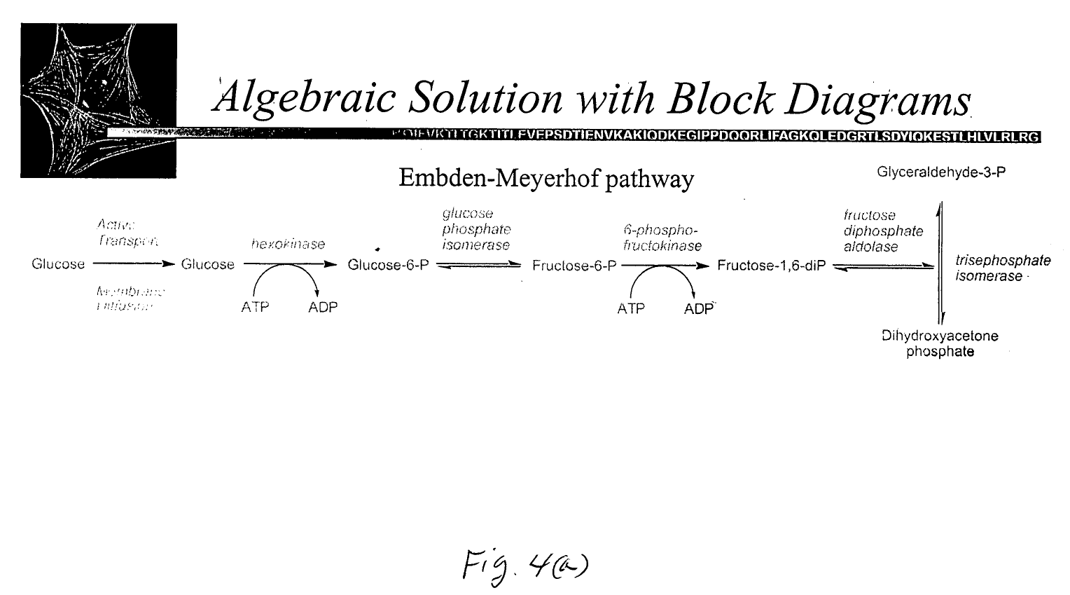

FIG. 3 depicts an illustration of the chromosome composition and generation of children for Systems Biology model generation. M represents the physiological unit operation set from which the model chromosome is built, where j is the specific unit operation element from the set and i is its position model chromosome. This assumes that all unit operations can be randomly distributed in each model position, although adaptations of the algorithm are obvious to those trained in the art that allow different sets in each position, such variations representing a Beysian approach. The overall model length is n, which is a constant in the example, but can also be variable between different chromosomes by allowing a null to be included in the set of unit operations. In this version of the algorithm, the relative fitness of the progeny is used to select which progeny will breed in the next generation. A random mutation is shown occurring at position 1 in child 4, which is also taken from the set of unit operations.

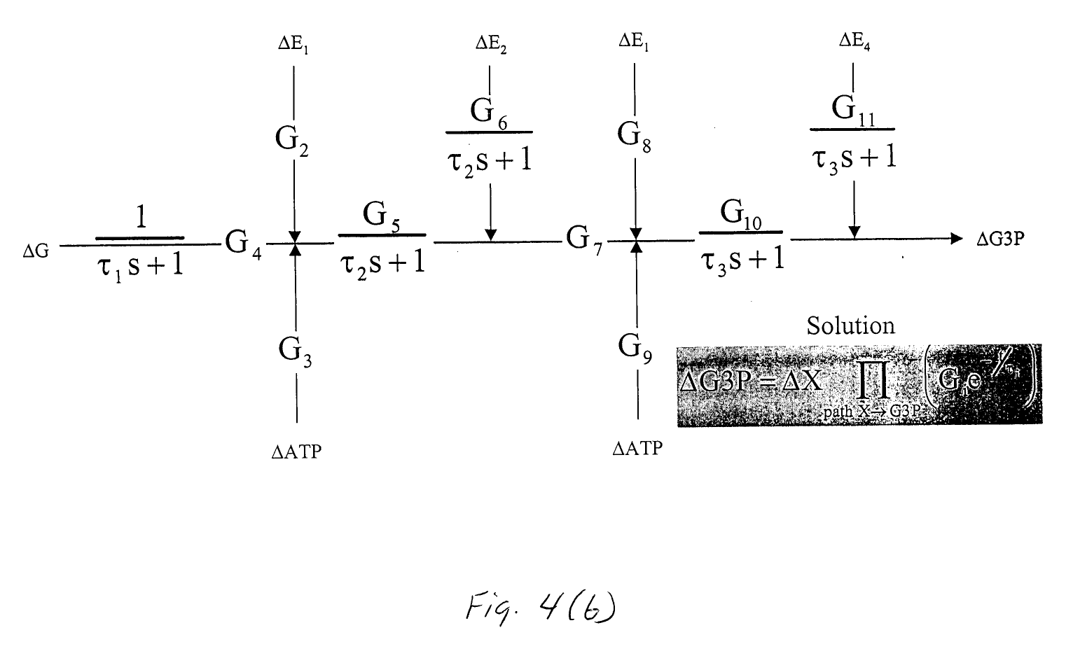

FIG. 4 depicts a schematic of a specific system of the invention. FIG. 4A depicts the Embden-Meyhoff system pathway with its components, and FIG. 4B depicts the pseudochromosome associated with this system.

### DETAILED DESCRIPTION

The present invention is directed to the use of evolutionary computational techniques, including but not limited to genetic algorithms (GAs), to derive and optimize mathematical models for a variety of systems. The systems can be any scientific system, such as biochemical/physiological systems, weather systems, traffic systems, economic and financial systems, market analysis systems, etc. The discussion below focuses on physiological systems.

A challenge in building systems biology models is the complexity of the underlying biochemistry. The larger the biological network, the harder the model is to formulate, the more complex (e.g. higher numbers of adjustable parameters), and the more models that can be proposed to fit the available data. Again, the bottleneck in the systems biology process is the human mind, or rather its ability to think of all the possible model permutations to describe a set of biological pathways.

Conversely, nature at its most basic biochemical level is not very diverse, exhibiting a high degree of functional conservation. There are numerous universal motifs in biochemistry, as all life is built on nucleic acids, proteins, lipids and carbohydrates. Ribosomes and translational codes are highly conserved within prokaryotic and eukaryotic genera. The primary energy generating process in both prokaryotes and eukaryotes is the proton motive force across a cell membrane. Nucleotide triphosphates are universally employed by all organisms as the energy shuttle between catabolic and anabolic metabolism. All known aerobic organisms contain a glycolysis (Embden-Meyerhof) pathway and tricarboxylic acid cycle. Transport of molecules through the cell membrane can be diffusional, facilitated, or active. Histidine protein kinase systems underlie many of the signal transduction processes in bacteria just as G-protein coupled receptor (GPCR) signal transduction is ubiquitous in most mammalian hormone and sensory processes. Further examples and sets are outlined below. The complexity in modeling biological systems, and underlying biodiversity, arises from how individual organisms piece these fundamental physiological units together to accomplish cellular tasks.

Thus, these biological pathways (sometimes referred to herein as “metabolic pathways” or “signaling pathways”) are made up of a series of discrete biological steps that can be classified and characterized, as well as organized temporally. For example, a particular pathway may comprise 7 steps in a particular and required temporal order, to achieve the biological goal. These seven steps may be individually selected from a large but finite list of physiological unit operations that define the basic biochemical levels as outlined below. For example, the individual physiological unit operations may comprise an enzymatic step (e.g. Michaelis Menton or Ping-Pong kinetics for proteases, kinases, etc.), transcription regulation (e.g. constitutive, repression, activatible, etc.), membrane transport (e.g. active, passive, facilitated, etc.), binding equilibria (e.g. affinity constants, etc.), diffusion (linear in the case of expression promoters on DNA or ribosomes acting on RNA; two-dimensional, as in the case of membrane proteins; and three-dimensional, as in the case of cytoplasmic or extracellular materials), enzyme regulation (allosteric, covalent modification, etc), convective transport (e.g., intertissue transport by blood or lymphatic circulation, movement of materials through the digestive tract, etc). That is, a physiological unit operation is an action that is done on a component of a system. Thus, the physiological unit operations represent a set of biochemicals that share one mathematical model that can be expressed via an equation or set of equations; the physiological unit operation is the mathematical model. Each member of the set differs from others by the identities of the other molecules that the member acts on (such as the substrate and product of an enzyme) and the values of the adjustable parameters of the model equation (such as the rate and Michaelis constants of an enzyme). Thus, any particular pathway or system may comprise any combination of these physiological unit operations in a particular order. For example, a pathway may be known to be a five step pathway, with seven different possible physiological unit operations, resulting in 7⁵ different possible combinations. However, biochemical pathways almost never operate in a straight line for more than a few conversion steps before hitting a branch point; in three dimensions, the system would have to generate and test more than 10¹² possible models, including redundancies, for the same 5 step pathway.

The present invention is directed to the use of computational methods to allow the generation, validation and/or refinement of mathematical models of particular systems, including biological pathways; essentially, the invention is an automated hypothesis generating and testing engine that directs the evolution of optimized models, particularly systems biology models. In preferred embodiments, these computational methods are evolutionary computational methods, described further below. This “in silico” testing allows the elimination of a majority of possible combinations, and thus allows a researcher to focus on combinations that explain empirical data to a significant degree. In some cases, a solution is found (there is convergence on a single solution), which then can be tested experimentally; alternatively, the process does not result in convergence, but several models either equally or closely fit the data, which are then tested; similarly, the process can be used iteratively to generate new models for testing.

The present invention finds use in three main modes: in a preferred embodiment, the invention finds use in validating mathematical models of existing systems. Secondly, the invention may be used to “fill in” missing steps in a pathway, by finding all possible physiological unit operations that will fit the empirical data and allowing a researcher to then focus on those. Finally, the invention may also be used in creating mathematical models, via a virtual “de novo” elucidation of one or more pathways.

The present invention can utilize a variety of artificial intelligence (Al) computational techniques to achieve these results. In particular, evolutionary computational techniques such as genetic algorithms, evolutionary programming, evolution strategies, classifier systems and genetic programming can all be used. For example, a genetic algorithm may be used in a preferred embodiment. GAs are described below, but in general, the physiological unit operations become pseudogenes which are arranged into pseudochromosomes to explain the data; for example, a five step pathway would be represented by a five pseudogene pseudochromosome, with the order and identity of the pseudogenes defining the pathway (see FIGS. 3 and 4). If a particular pseudochromosome does not fit the data, it can be “crossed” or “recombined” with other pseudochromosomes and evaluated for fitness. That is, pairs of psuedochromosomes are selected as “parents” (generally those with the best fitness rating, e.g. ability to fit the data, sometimes referred to herein as “first order pseudochromosomes”), and these pairs are mated using various techniques to generate “children” or “second order” (or higher) pseudochromosomes. These “children” are evaluated against the empirical data and the process is iterated to produce better “fit” pseudo-chromosomes. Higher order chromosomes are used above to refer to dimensionality of the chromosome rather than the quality of the chromosome. This process is repeated until a globally optimized solution pseudochromosome, representing a particular set of physiological unit operations in a particular (temporal) order, is found. Alternatively, in some cases the algorithms are not run to convergence; either because multiple solutions can be found, or because convergence is not desired. In these cases, multiple experiments may be run. In additional embodiments, other Al techniques may be used, as is further described below.

Thus, this approach consists of creating an object-oriented library of physiological unit operations, as outlined below and including enzyme kinetic operations, membrane transport operations, and binding equilibria models, etc. These physiology unit operations become the pseudogenes that comprise the genetic algorithm pseudochromosome (FIGS. 3 and 4). The psuedochromosome itself is the model, determining the best order with which to string the unit operations together. In the schematic shown, this psuedochromosome represents a linear arrangement of unit operations. However, the actual psuedochromosomes used may have higher order dimensionality to accommodate branches in biochemical networks.

Thus, this approach uses a fitness function to direct the evolution of the psuedochromosome to better models. The fitness function may consist of several parts. The first part may be a strict life/death decision based on validated pathways taken from the literature. The second part may be a goodness of fit to the proteomic and metabolomic data already generated. The final part consists of user-definable limiting assumptions. An example of one such limiting assumption is the requirement that the model be stable (i.e., exhibit a single single steady state solution for a given set of inputs). Another example is to add a penalty for the fitness function for pseudochromosomes containing higher numbers of psuedogenes.

Optimized models can then be used for several purposes. Where there is failure to converge to a single model, the model(s) can be manipulated in silico to define additional empirical validation experiments that can delineate between the best models. Sensitivity analysis of the final surviving model(s) is then conducted to determine the best diagnostic biomarkers and points of therapeutic intervention.

In an embodiment of the present invention, the physiological unit operations may consist of sets of dimensionless differential equations, with the solution of each pseudochromosome model consisting of a numerical integration in the time domain.

In another embodiment of the present invention the physiological unit operations may be converted to difference equations by the subtraction of a control or steady-state form of the model. In this embodiment of the invention, the resulting difference equations may be used directly with differential display data types in testing the goodness of fit.

In another embodiment of the present invention, the physiological unit operations may be linearized, either by conversion to difference equations or by methods such as Taylor series expansion of non-linear terms. The resulting linearized equations are then transformed to other mathematical domains, such as the Laplace domain (or other domains, as outlined below), to allow faster solution of the models through algebraic manipulation in the pseudochromosomes.

Because these models are rooted in the fundamental differential equations representing physiological unit operations, they are also predictive tools, allowing the researcher to alter parameters and play “what if” scenarios. Where laboratory data is incomplete, such that multiple models provide equally good fits, the ability to tweak protein or metabolite concentrations in silico allows the researcher to quickly identify new hypotheses and design definitive experiments to distinguish the true underlying physiological mechanism. Once a physiological model is validated, it can then be used to select the best targets for therapeutic intervention or diagnosis, by sensitivity analysis in silico, thus guiding the best course of treatment or the selection of target(s) for new drug discovery.

Accordingly, the present invention provides methods comprising providing a plurality of unit operations that represent all or subset of all actions that can be done on system components. By “system” in this context means the system for which a mathematical model is desired. As will be appreciated by those in the art, the system can be virtually any system which includes discrete unit operations put together in complicated and generally non-intuitive patterns. Suitable systems include, but are not limited to, biological systems, traffic systems, weather systems, traffic systems, economic and financial systems, market analysis systems, etc. The remaining discussions will focus on biological systems, but this is not meant to limit the scope of the invention in any manner.

A plurality of unit operations represent the actions of the set of system components. By “plurality” herein is meant at least two “Unit operations” or “physiological unit operations” (when the system is biological) are defined consistent with the definition common to chemical engineering [McCabe, W. L. and J. C. Smith, Unit Operations of Chemical Engineering, 3rd edition (McGraw-Hill, NY, 1976)]. Although the number of individual process systems is great, each one can be broken down into a series of steps, called operations, each operation appearing in many process systems. Each individual operation has common techniques and is based on the same scientific principles. By defining these principles and incorporating them into a common mathematical representation the individual operation becomes a unit operation. A number of scientific principles and techniques are basic to the treatment of the unit operations. Some are elementary physical and chemical laws, such as conservation of mass and energy, physical and chemical equilibria, kinetics, and certain properties of matter. As discussed herein, biochemical and physiological pathways are considered process systems consisting of individual components. As discussed herein, the components of the pathway include, but are not limited to, nucleic acid (DNA, RNA (including mRNA, tRNA, snRNA, siRNA, etc.), proteins (including binding proteins, enzymes, peptides, etc.), carbohydrates, lipids, and metabolites. In general, the discussion below is centered on protein components, but this is not meant to limit the scope of the invention in any way.

These biochemical properties or characteristics of individual components can include, but are not limited to, enzyme kinetic equations such as Michaelis Menton kinetic equations, membrane transport equations, binding equilibria equations, diffusional kinetics (e.g. a one dimensional diffusion system could be a DNA binding protein on a chromosome; a two dimensional analysis could be a receptor in a cell membrane; and a three dimensional analysis could be intra- or extracellular diffusional kinetics), convective transport either within a cell or tissue, or between cells and tissues (such as chemicals transported with the circulatory, lymphatic, or cerebral spinal fluid systems), regulatory mechanisms (such as allosteric or covalent modification of enzymes or transmembrane proteins to affect their function), etc. Preferred equations include, but are not limited to, unimolecular chemical equilibria, biomolecular equilibria, enzyme-mediated equilibria, Michaelis-Menton kinetics, biomolecular enzyme reactions, Michaelis-Menton enzyme kinetics with allosteric upregulation, and Michaelis-Menton enzyme kinetics with allosteric repression, as are outlined in the Examples. From these biochemical parameters, physiological unit operations are generated; the physiological unit operations are the mathematical equations representing the underlying scientific principles common to a specific operation common to more than one process system. The unit operations reflect actions performed on physical entities, such as chemical conversions, adsorption/desorption, diffusion, and transport of molecules. In financial and marketing contexts unit operations reflect actions performed on objects (e.g., materials and money), which can be exchanged, transported, or converted to other objects (e.g., the exchange of cash for food at a store). In the context of traffic, the objects may be people and/or their vehicles upon which the actions of traffic signals and the constraints of storage (parking lots), and flow channels (roads) operate.

In a preferred embodiment, the physiological unit operations describe proteins; that is, they represent some or all of the biochemical actions that can be done on or with proteins. By “protein” herein is meant at least two amino acids linked together by a peptide bond. As used herein, protein includes proteins, oligopeptides and peptides. The peptidyl group may comprise naturally occurring amino acids and peptide bonds, or synthetic peptidomimetic structures, i.e. “analogs”, such as peptoids (see Simon et al., PNAS USA 89(20):9367 (1992)). The amino acids may either be naturally occurring or non-naturally occurring, depending on the use, as is more fully described below. For example, when candidate bioactive (e.g. drug) agent screening is done, the candidate agents may be synthetic peptides. The side chains may be in either the (R) or the (S) configuration. In a preferred embodiment, the amino acids are in the (S) or L-configuration.

In a preferred embodiment, the physiological unit operations describe enzymes; e.g. the unit operations describe all or some of the procedures that can be done on or with enzymes. As will be appreciated by those in the art, there are a wide variety of enzymes that are involved in metabolic pathways, including hydrolases such as proteases, carbohydrases, lipases; isomerases such as racemases, epimerases, tautomerases, or mutases; transferases, kinases and phophatases. Preferred enzymes include those that carry out group transfers, such as acyl group transfers, including endo- and exopeptidases (serine, cysteine, metallo and acid proteases); amino group and glutamyl transfers, including glutaminases, γ glutamyl transpeptidases, amidotransferases, etc.; phosphoryl group transfers, including phosphotases, phosphodiesterases, kinases, and phosphorylases; nucleotidyl and pyrophosphotyl transfers, including carboxylate, pyrophosphoryl transfers, etc.; glycosyl group transfers; enzymes that do enzymatic oxidation and reduction, such as dehydrogenases, monooxygenases, oxidases, hydroxylases, reductases, etc.; enzymes that catalyze eliminations, isomerizations and rearrangements, such as elimination/addition of water using aconitase, fumarase, enolase, crotonase, carbon-nitrogen lyases, etc.; and enzymes that make or break carbon-carbon bonds, i.e. carbanion reactions. Specific unit operations are known for a wide variety of these enzymes, particularly proteases such as serine, cysteine, aspartyl and metalloproteases, including, but not limited to, trypsin, chymotrypsin, and other therapeutically relevant serine proteases such as tPA and the other proteases of the thrombolytic cascade; cysteine proteases including: the cathepsins, including cathepsin B, L, S, H, J, N and O; and calpain; metalloproteinases including MMP-1 through MMP-10, particularly MMP-1, MMP-2, MMP-7 and MMP-9; and caspases, such as caspase-3, -5, -8 and other caspases of the apoptotic pathway, and interleukin-converting enzyme (ICE). Suitable enzymes are listed in the Swiss-Prot enzyme database. The enzymes may be naturally occurring or variant forms of the enzymes. For example, many disease states are due to variant enzymes.

In a preferred embodiment, the proteins are binding proteins, and the physiological unit operations generally comprise binding equilibria equations and affinity constants. For example, preferred binding proteins crucial in a wide variety of signaling pathways are pairs of ligands and cell surface receptors (some of which are also enzymes, such as kinases). Suitable ligands include, but are not limited to, all or a functional portion of the ligands that bind to a cell surface receptor selected from the group consisting of insulin receptor (insulin), insulin-like growth factor receptor (including both IGF-1 and IGF-2), growth hormone receptor, glucose transporters (particularly GLUT 4 receptor), transferrin receptor (transferrin), epidermal growth factor receptor (EGF), low density lipoprotein receptor, high density lipoprotein receptor, leptin receptor, estrogen receptor (estrogen); interleukin receptors including IL-1, IL-2, IL-3, IL-4, IL-5, IL-6, IL-7, IL-8, IL-9, IL-11, IL-12, IL-13, IL-15, and IL-17 receptors, human growth hormone receptor, VEGF receptor (VEGF), PDGF receptor (PDGF), transforming growth factor receptor (including TGF-α and TGF-β), EPO receptor (EPO), TPO receptor (TPO), ciliary neurotrophic factor receptor, prolactin receptor, and T-cell receptors. In particular, hormone ligands are preferred. Hormones include both steroid hormones and proteinaceous hormones, including, but not limited to, epinephrine, thyroxine, oxytocin, insulin, thyroid-stimulating hormone, calcitonin, chorionic gonadotropin, cortictropin, follicle-stimulating hormone, glucagon, leuteinizing hormone, lipotropin, melanocyte-stimutating hormone, norepinephrine, parathryroid hormone, thyroid-stimulating hormone (TSH), vasopressin, enkephalins, seratonin, estradiol, progesterone, testosterone, cortisone, and glucocorticoids and the hormones listed above. Receptor ligands include ligands that bind to receptors such as cell surface receptors, which include hormones, lipids, proteins, glycoproteins, signal transducers, growth factors, cytokines, and others.

In a preferred embodiment, the physiological unit operations involve carbohydrates. By “carbohydrate” herein is meant a compound with the general formula Cx(H₂O)_{y}— Monosaccharides, disaccharides, and oligo- or polysaccharides are all included within the definition and comprise polymers of various sugar molecules linked via glycosidic linkages. Particularly preferred carbohydrates are those that comprise all or part of the carbohydrate component of glycosylated proteins, including monomers and oligomers of galactose, mannose, fucose, galactosamine, (particularly N-acetylglucosamine), glucosamine, glucose and sialic acid, and in particular the glycosylation component that allows binding to certain receptors such as cell surface receptors. Other carbohydrates comprise monomers and polymers of glucose, ribose, lactose, raffinose, fructose, and other biologically significant carbohydrates.

In a preferred embodiment, the physiological unit operations involve lipids. “Lipid” as used herein includes fats, fatty oils, waxes, phospholipids, glycolipids, terpenes, fatty acids, and glycerides, particularly the triglycerides. Also included within the definition of lipids are the eicosanoids, steroids and sterols, some of which are also hormones, such as prostaglandins, opiates, and cholesterol. For example, during apoptosis and cell death, there is a significant alteration in the polarity of lipids within cell membranes, with the asymmetrical distribution of inner and outer layers of the bilayer that exists during healthy cell life becoming more symmetrical (e.g. the lipids “flip” towards equilibrium between the bilayers).

A preferred physiological unit operation describes enzymes, and comprises a Michaelis Menton equation:

$$\frac{ⅆ}{ⅆ t} ( Substrate [ t ] ) = - \frac{k Enzyme [ t ] Substrate [ t ]}{( {K}_{m} + Substrate [ t ] )} \qquad ( 1 )$$

The unit operations are generally organized into a database. As will be appreciated by those in the art, there are a variety of ways to do this. In one embodiment, the physiological unit operations are in the form of systems of differential equations in the time domain, and are stored as such. However, for a variety of reasons, this is not preferred; integration is time consuming as well as computationally intense. In addition, the use of differential equations requires the use of an integration tool, and the resulting system models can usually only be solved numerically. Because of the vastly different time scales involved in human physiology—i.e., cancerous tumors develop over the course of years, while metabolic conversions may occur in seconds—many of these models will be stiff differential equations. The efficient solution of a system of stiff differential equations requires a predictor-corrector integrator designed for stiff differential equations. The open source code for several such stiff integrators (e.g., GEAR and RODAS) have been thoroughly reviewed in the literature. The Rosenbrock methods (RODAS) appear to be particularly well suited to the solution of chemical reaction equations and biochemical systems and are integrated in the Berkeley Madonna package, which comes with several pre-cast biochemical pathway examples. A differential equation approach, however, generates large numbers of adjustable parameters, allowing more models to fit a specific data set. Finally, distinguishing between models will require higher precision, quantitative, absolute expression data than is currently available in the “OMICS” databases. Most of the data currently in the “OMICS” databases is of the differential expression type (i.e., GeneChip™, proteomics data, ICAT™, and SIR metabolomic data), which means that few parameters, like concentrations and rate constants, are known in an absolute sense.

Thus, in a preferred embodiment, the physiological unit operations are transformed to another mathematical domain. Generally, any linear transform converting a differential equation into a domain solveable with algebraic operations may be used. As will be appreciated by those in the art, there are a wide variety of suitable transforms available, including, but not limited to, Laplace transforms, Buschman transforms, Fourier transforms (of which there are a variety, such as discrete time (DTFT), continuous time (CTFT), fast (FFT), etc.), Fourier-Stieltjes Transform, G-Transform, H-Transform, Hadamard Transform, Hankel Transform, Hartley Transform, Hough Transform, Kontorovich-Lebedev Transform, Mehler-Fock Transform, Meijer Transform, Narain G-Transform, Operational Mathematics, Radon Transform, Stieltjest Transform, W— Transform, Wavelet Transform, Z-Transform, etc.

However, when transforming differential equations into algebraic operations, one limitation is that these transforms (e.g. the Laplace domain) requires the use of linearized equations. Thus, many physiological unit operations are expressed as non-linear differential equations, and must be linearized. Linearization can be done in a wide variety of ways, as will be appreciated by those in the art. In a preferred embodiment, more fully outlined below, any number of assumptions may be made about the magnitude of certain coefficients to disregard the non-linearity. Alternatively, expansions may be done that results in a sum of linear portions, followed by the elimination of some of the higher-order terms; for example, a Taylor series expansion may be done.

In a preferred embodiment, linearization is done using common biological assumptions or by using difference equations in either the time of the Laplace domain (e.g. where the unit operations are converted to represent deviations from a control). The use of difference equations has the advantage of mapping more directly to current “OMICS” data, which is typically expressed as changes in expression from that of a control.

The equation for Michaelis-Menton kinetics can be used to illustrate these alternative approaches. In differential equation form this is:

$$\frac{ⅆ}{ⅆ t} ( Substrate [ t ] ) = - \frac{k Enzyme [ t ] Substrate [ t ]}{( {K}_{m} + Substrate [ t ] )} \qquad ( 1 )$$

Unless the form of the solution is known, it is not possible to directly take the Laplace transform of this equation, as it is nonlinear. Although, it is possible to linearize the equation at one of its two extremes. In the case where Substrate[t]>>Km, we get the linear differential equation (2) and its Laplace transform (3):

$$\frac{ⅆ}{ⅆ t} ( Substrate [ t ] ) = - \frac{k Enzyme [ t ]}{{K}_{m}} \qquad ( 2 )$$

$$Substrate [ s ] = - ( \frac{k}{{K}_{m} s} ) Enzyme [ s ] \qquad ( 3 )$$

The challenge then remains about what to do when the substrate concentration is not in great abundance. Here the concept of the difference equation is of utility. If we define a the deviation of the substrate (ΔS[t]) and enzyme (ΔE[t]) concentrations over time from that of a control state (c):

ΔS[t]=Substrate[t]−Substrate[c] (4)

ΔE[t]=Enzyme[t]−Enzyme[c] (5)

then we get the derivative relationship:

$$\frac{ⅆ}{ⅆ t} ( Substrate [ t ] ) = \frac{ⅆ}{ⅆ t} ( Δ S [ t ] ) + \frac{ⅆ}{ⅆ t} ( Substrate [ c ] ) \qquad ( 6 )$$

Substitution of equations 4, 5, and 6 into Michaelis-Menton equation (1) yields:

$$\begin{matrix}\frac{ⅆ}{ⅆ t} ( Δ S [ t ] ) = \frac{k Enzyme [ c ] Substrate [ c ]}{( {K}_{m} + Substrate [ c ] )} - \\ \frac{k ( Δ E [ t ] + Enzyme [ c ] ) ( Δ S [ t ] + Substrate [ c ] )}{( {K}_{m} + Δ S [ t ] + Substrate [ c ] )}\end{matrix} \qquad ( 7 )$$

If we assume that ΔS[t] deviates only slightly from (Kₘ+Substrate[c]), then the first two terms cancel to a first approximation. If we further assume that the second order deviation (ΔS[t] ΔE[t]) can be neglected, then we get the linearized deviation equation (8) and its Laplace transform (9), which allows us to relate a change in enzyme level to a change in substrate level to a first approximation.

$$\begin{matrix}\frac{ⅆ}{ⅆ t} ( Δ S [ t ] ) + \frac{k Enzyme [ c ]}{( {K}_{m} + Substrate [ c ] )} Δ S [ t ] + \frac{k Substrate [ c ]}{( {K}_{m} + Substrate [ c ] )} Δ E [ t ] = 0 & ( 8 ) \\ Δ S [ s ] = \frac{- \{ \frac{Substrate [ c ]}{Enzyme [ c ]} \}}{\{ \frac{( {K}_{m} + Substrate [ c ] )}{k Enzyme [ c ]} \} s + 1} Δ E [ s ] & ( 9 )\end{matrix}$$

Taking the inverse Laplace transform of equation 9 yields both steady-state and dynamic solutions that relate changes in the enzyme level (as determined from gene or protein expression data) to changes in the substrate level (as determined from proteomic or metabolomic data). The numerator in equation 9 suggests that upregulation of the enzyme concentration will translate into a down-shift in substrate concentration over that of the control state with a proportionality constant (G) of:

$$G = \frac{Substrate [ c ]}{Enzyme [ c ]} \qquad ( 10 )$$

When the substrate concentration is low relative to the enzyme concentration, very little change will be seen in the substrate concentration for large changes in enzyme concentration. An example of this situation might be protein kinase activity. However, when the substrate concentration is high relative to the enzyme concentration, small changes in the enzyme concentration will produce disproportionately larger changes in the substrate concentration. Small molecule metabolism (e.g., cholesterol degradation or sugar metabolism) might provide an example. Furthermore, the denominator of equation 9 suggests that the substrate will approach its new concentration as a simple exponential decay with a time constant (τ) of;

$$τ = \frac{( {K}_{m} + Substrate [ c ] )}{k Enzyme [ c ]} \qquad ( 11 )$$

and the higher the substrate concentration in the control sample, the slower it will approach its new value. The real power of this approach, however, is that it allows us to work directly with differential display data—by far the most common form of “OMICS” data—because it eliminates the need for absolute determination of the gene, protein, and metabolite concentrations. Both G and τ can be determined by curve fit to differential display data. Yet, given independent measurements of the rate (k) and Michaelis (Kₘ) constants, the actual substrate and enzyme concentrations can be still be determined by simultaneous solution of equations 10 and 11. Hence, as additional absolute information is acquired it is possible to return directly to a more standard differential equation based model.

Additional examples of preferred physiological unit operations are shown in the Examples, including, but not limited to, unimolecular chemical equilibria, biomolecular equilibria, enzyme-mediated equilibria, Michaelis-Menton kinetics, biomolecular enzyme reactions, Michaelis-Menton enzyme kinetics with allosteric upregulation, and Michaelis-Menton enzyme kinetics with allosteric repression

Once the database of preferably algebraic physiological unit operations are stored, a first hypothetical model is generated using all or a set of the physiological unit operations. In genetic algorithm approaches, several additional hypothetical models may also be formulated using random or semi-random combinations of physiological unit operations. Each of these models is then tested for fitness against exisiting “omics” data and any other model constraints. The best models are those that satisfy all the constraints on the system and provide the best fit of the “omics” data. The adjustable model parameters (see definition below) are gleaned from the available “omics” data, literature on similar unit operations in similar systems, or intuition. The best values for the adjustable model parameters of the model may be determined by least squares or least median squares curve fits where the available “omics” data is overspecified. Alternatively, where the available data is just sufficient or insufficient, some of the adjustable model parameters must be estimated using researcher judgement.

The starting set of physiological unit operations to be considered can thus be chosen in a variety of ways. As outlined above, the entire possible set can be used, or a subset. The subset(s) can chosen in a wide variety of ways. In some embodiments, it may be known that a particular signaling pathway does not contain certain steps; for example, an entirely intracellular pathway may not contain a cellular membrane transport step. Thus, the starting set of physiological unit operations may remove those particular physiological unit operations from the operation. Similarly, some or part of pathway may be known, and thus the physiological unit operations for these parameters are specifically included. Alternatively, it may be desirable to randomly select a subset of physiological unit operations for consideration into a particular pathway.

The set of unit operations incorporated in a system model also defines a set of possible model parameters. By “model parameters” herein is meant the set of variables (e.g., rate constants, affinity constants, transport coefficients, phase or chemical equilibrium constants, etc.) that are involved in a particular system model. Preferably, all possible model parameters are defined or deduced from empirical “omics” data collected on the system. Where data is incomplete, the model parameters must be estimated from similar biochemical systems or the researchers intuition.

This starting set of physiological unit operations can be combined to form a first hypothetical mathematical model of the system. As will be appreciated by those in the art, this can be done in a wide variety of ways, including randomly, directed or computationally, including statistically, and may depend on the algorithm used.

In some instances, for example when genetic based artificial intelligence algorithms, such as genetic algorithms, are used, the pseudogenes are combined into pseudochromosomes, which form the first hypothetical mathematical model. As will be appreciated in the art, there are a wide variety of ways to create the first pseudochromosomes. In one embodiment, all possible combinations are made; that is, every physiological unit operation is put at every position and in every order to form the starting set of parent pseudochromosomes. This allows an exhaustive search of all possible models, however, is generally not preferred because of the computational time involved in conducting such an exhaustive search. In some embodiments, some positions within the pseudochromosome are “fixed□ as particular physiological unit operations. That is, a pathway may be known to contain a starting membrane transport parameter. Similarly, in the case of pathway “branch” points, certain positions within the pathway may be known to lead to higher order possibilities (e.g. non-linear pseudochromosomes), and thus can be fixed as branch points. In some embodiments, existing models are used to create the first sets of pseudochromosomes. Alternatively, the first pseudochromosomes are generated randomly.

Once the first hypothetical model is generated, one or more artificial intelligence (Al) algorithms is applied to the model. As will be appreciated by those in the art, there are a wide variety of suitable algorithms that can be utilized in the present invention, including both deterministic and non-deterministic methods. In general, deterministic methods are preferred in most instances, as some convergence on a single solution is desired. However, as will be appreciated by those in the art, many of the techniques outlined below are non-deterministic. In these cases, a fitness function or selection pressure may be used to drive a solution towards convergence. In addition, as further outlined below, it may be desirable in some cases to change the fitness function and re-run the calculations, one or more times, to generate a set of possible solutions. Similarly, there are methods that allow the identification of local minima, which also may be useful, rather than a single global optimum solution.

In a preferred embodiment, the Al is a genetic or evolutionary algorithm. In general, an evolutionary algorithm (EA) applies the principles of evolution found in nature to find one or more solutions, preferably a single optimal solution. In general, these EAs rely on a number of parameters. EAs normally include deterministic functions. However it is also possible to incorporate non-deterministic elements into EAs, where multiple outcomes are pooled to yield a average result. In one embodiment, Monte Carlo methods are used to pool results from non-deterministic models. In another embodiment, the EA includes random sampling as a non-deterministic method (e.g. different solutions will be reached on different runs), in the absence of selective pressure such as a fitness function, described more fully below. EAs generally initiate and maintain a population of candidate solutions rather than a single solution. This allows a wider sampling of search space, and helps the EA avoid becoming “trapped” at a local optimum rather than a global optimum. EAs can also include the use of “mutation”, wherein the EA periodically makes random changes or mutations to the current population. EAs also frequently rely on the use of cross-over (particularly GAs) to combine elements of existing solutions to create new solution(s). Sometimes these crossovers are weighted with more favorable elements of the solution being given priority in the crossover. For example, a crossover weighting towards having certain unit operations being present (e.g. an enzymatic unit operation) may be done Finally, the EAs incorporate the use of a “selection” or “fitness function” to direct the evolution of the solution(s).

As outlined herein, there are a variety of evolutionary computational techniques that can be used, such as genetic algorithms, evolutionary programming, evolution strategies, classifier systems and genetic programming can all be used. See for example Genetic Programming III: Darwinian Invention and Problem Solving, by John R. Koza et al., Morgan Kaufmann Publishers; Genetic Algorithms: in Search, Optimization and Machine Learning, Goldberg, 1989, Addison-Wesley Publishing Co.; Beset, D. H., Object-oriented implementation of numerical methods: An introduction with Java and Smalltalk (Morgan Kaufmann, San Francisco, 2001), all of which are hereby incorporated by reference in their entirety. In general, the operations of an Al algorithm mimic biological functions, including reproduction, crossover (sexual recombination), mutation, and architecture-altering operations patterned after gene duplication and gene deletion in nature. To distinguish from the “actual” biological systems outlined herein, these functions will all be referred to as “psuedo” functions, despite their use in computer literature without this prefix.

The main generational loop of a run of genetic programming consists of the fitness evaluation, Darwinian selection, and the pseudogenetic operations. Each individual hypothetical mathematical model in the population is evaluated to determine how fit it is as compared to the empirical data and other constraints. Models are then probabilistically selected from the population of models based on their fitness to participate in the various genetic operations, with reselection allowed. While a more fit model has a better chance of being selected, even individuals known to be unfit are optionally allocated some trials in a mathematically principled way.

In a preferred embodiment, the Al algorithm includes a pseduomutation operation. This can be done in a variety of ways, generally by randomly selecting a particular parameter (e.g. a pseudogene) and randomly changing it to another. In general, this asexual pseudomutational operation is typically performed sparingly (with a low probability in each recombination event. The exact rate of mutation must be empirically optimized for each application, but is generally less than 10% and more typically less than 1%.

In a preferred embodiment, the Al algorithm includes a pseudocrossover (e.g. sexual recombination) operation. In the pseudocrossover, or pseudosexual recombination operation, two parental models are probabilistically selected from the population based on fitness. The two parents participating in pseudocrossover may be of the same or different sizes and shapes. A pseudocrossover point is randomly chosen in the first parent and a pseudocrossover point is randomly chosen in the second parent. Pseudocrossover is the predominant operation in genetic programming (and genetic algorithm) work and is performed with a high probability (say, 85% to 90%).

In one embodiment, the Al algorithm includes a pseduoreproduction operation, which copies a single individual model, probabilistically selected based on fitness, into the next generation of the population. In another embodiment, offspring (psuedochromosomes resulting from crossover operations and mutations) are ranked against members of the parent population based on their scores in the fitness function and replace less fit members of the parent population in subsequent crossover and mutation rounds.

In addition, in a preferred embodiment, one or more architecture-altering operations are used. While simple signaling pathways may be represented by a single linear model, more commonly, the pathways may comprise subpathways, iterations, loops (including feedback loops), branch points, recursions, etc. If a human user is trying to solve an engineering problem, he or she might choose to simply prespecify a reasonable fixed architectural arrangement for all programs in the population (i.e., the number and types of branches and number of arguments that each branch possesses). Genetic programming can then be used to evolve the exact sequence of primitive work-performing steps in each branch.

However, sometimes the size and shape of the solution is the problem. In this instance, genetic programming is capable of making all architectural decisions dynamically during the run of genetic programming. Genetic programming can use architecture-altering operations to automatically determine mathematical architecture in a manner that parallels gene duplication in nature and the related operation of gene deletion in nature. Architecture-altering operations provide a way, dynamically during the run of genetic programming, to add and delete branches to individual models. These architecture-altering operation quickly create an architecturally diverse population containing models with different numbers of branch points, iterations, loops, recursions, etc., and, also, different hierarchical arrangements of these elements. Models with architectures that are compatible with the empirical data tend to grow and prosper in the competitive pseudoevolutionary process, while models with inadequate architectures will tend to be disfavored under the fitness function. Thus, the architecture-altering operations relieve the human user of the task of prespecifying program architecture.

As is known in the art, there are several different architecture-altering operations. They are each applied sparingly during the run (for example with a probability of less than 10% and more typically less than 1% on each generation).

In a preferred embodiment, a genetic algorithm is used, as outlined herein.

In a preferred embodiment, with many or all of the Al algorithm, a fitness function is used to select between alternative members of the solution set to find the optimum solution. The fitness function is used to direct the evolution of the model (e.g. the pseudochromosome when a GA is used) and to allow non-deterministic methods to converge. In general, the fitness function may consist of several parts. In one embodiment, the fitness function may include a strict life/death decision based on validated pathways taken from the literature. That is, global solutions may be known to contain or avoid certain physiological unit operations or parameters, or dictate or preclude different temporal orders. For example, a membrane transport step may be known to be required, and thus any possible solutions which do not contain this physiological unit operation are eliminated. Similarly, certain enzymatic steps must be in certain orders: the enzymatic conversion of a substrate cannot come before its creation, etc. These “rules” are generally drawn from the literature or thermodynamic principles. “Constraints-based” modeling approaches, such as those described by [Proc. National Acad. Sci. (USA) 95:4193-4198, 1998; Biotechnology Progress, 15:296, 1999; J. theor. Biol., 203:229-249, 2000] can be incorporated in the fitness function. However, it is also possible to create these “per se” rules, run the system and evaluate the output. For example, when it is not known whether a membrane transport function is involved, running the system with either rule and fitting it to the known empirical data can help elucidate the model. In another embodiment, the fitness function may be a goodness of fit measure to empirical data, e.g. the OMICS expressional genomic, proteomic, and metabolomic data already generated. At a simplistic level the fitness function can be considered as a statistical goodness of fit measurement in a curve fit to experimental data. In another embodiment, the fitness function may include user-definable limiting assumptions, such as a test for multiplicity of steady-states or violation of physical and chemical laws (i.e. constrants). It can be argued that multiple steady-states (i.e., more than one possible physiological state being possible for a fixed set of growth conditions) are inefficient, like futile cycles, and would have been selected against in nature. In a preferred embodiment the fitness function includes more than one or all of the above described embodiments.

The fitness function is generally applied to each proposed member of the solution set. For example in GAs it is applied to each psuedochromosome in the population. Psuedochromosomes exhibiting the best numerical scores in the fitness function survive to the next generation. In one embodiment any child psuedochromosome exhibiting a fitness function score better than the worst member of the parent population replaces the worst parent psuedochromosome in the population. In another embodiment, any psuedochromosome exhibiting better than a threshold fitness score is added to the population, thus the number of psuedochromosomes in the population increases with the number of iterations. In a preferred embodiment, the threshold score is adjusted over time to eliminate poorly performing members of the population.

Once an Al algorithm (again, preferably a GA) is chosen, it is applied against the first hypothetical model. For example, in the case of GAs, the first model comprising the parent pseudochromosomes are then utilized in a GA to generate “child” pseudochromosomes, which comprise the second hypothetical model. Similarly, other algorithms result in alternate second hypothetical models. This second model is then compared against the empirical data, and the process is iterated until either a global solution is found or a defined set of possible solutions is reached, as is more fully outlined below.

Accordingly, mathematical models generated using the processes of the invention are compared against empirical data. As will be appreciated by those in the art, the empirical data that can be used in the present invention can comprise virtually any experimental data. The data can be quantitative and/or qualitative data, including “absolute” or “difference” data.

In a preferred embodiment, the first set of empirical data comprises a set of difference data such as is usually generated during many of “OMICS” evaluations. A critical issue surrounding most modern biological data collection methods is that they only provide a measure of the differences between samples. For example, GeneChip™ data utilizes reverse transcription and quantitative polymerase chain reaction (PCR) to provide a measure of up and down regulation of specific mRNAs compared to a control array. Protein expression, such as in 2-D gel electrophoresis experiments, provides a relative measure of the abundance of each protein based on the quantity of stain accumulated at a spot in the gel. The recent invention of mass spectrometer-based differential display techniques, such as isotope coded affinity tags (ICAT™)¹ and isotope differentiated binding energy shift tags (IDBEST™),² allows the direct quantitative comparison of relative protein expression between two or more samples based on the ratio of stable isotopes. Stable isotope ratio methods are also being used to provide quantitative comparison of relative metabolite concentrations between two samples by nuclear magnetic resonance (NMR)³ and mass spectrometry (MS).⁴ These differential display methods result in ratiometric comparisons rather than absolute comparisons traditionally available in chemistry and other scientific disciplines. The advantage of these differential display methods is that they are relatively inexpensive, quick, and accurate (<10% standard deviation). The disadvantage, from a modeling prospective, is that they fail to provide absolute quantitative concentration and rate constant information. 1 Aebersold, R. H., et al., WO 00/11208 (Mar. 2, 2000). 2 Schneider, L. V. et al., WO 01/49951 (Aug. 29, 2002); Hall, M. P. et al., paper presented at the Amer. Soc. Mass Spectro. 50^{th} Annual Meeting, Orlando, Fla. (June, 2002). 3 Gonzalez, B., Piotto, M., and G. Huber, WO 02/31523A1 (Apr. 18, 2002). 4 Schneider, L. V. et al., WO 00/63683 (Oct. 26, 2000).

In a preferred embodiment, several different types of empirical data are used in the evaluation, and preferably all the relevant data is used. As outlined herein, there are a wide variety of different types of OMICS data (see Lederberg, supra). In preferred embodiments, as many different types of data as are obtainable are evaluated against the hypothetical models. In general, it may be useful to “weight” these different types of data differently, as it is known that different types of data have different levels of correlation to each other. For example, mRNA expression levels do not always correlate to protein expression levels, with the latter being probably more physiologically relevant in many circumstances. Accordingly, “weighting” the protein data greater than the expression data may be done. In a preferred embodiment the physiological unit operations used in the models are difference equations (or a mathematical transformation of difference equations). By using difference equations it is possible to directly use differential display data directly in the fitness function.

In an embodiment that does not utilize difference equations for the physiological unit operations, an assumption of the absolute value or absolute empirical measurement must be made so that differential display data may be converted to absolute values for use in the fitness function.

These processes are iterated towards a convergence. As will be appreciated by those in the art, the system may be “stopped” at any number of places. In a preferred embodiment, the fitness functions utilized allow convergence on a global solution; that is, one model is evolved that matches one or more sets of empirical data, which then is subsequently used as outlined below. This global solution then allows the generation of additional local solutions as outlined below, if necessary or desired. Alternatively, absolute convergence on a single solution may not be either possible or desired.

In a preferred embodiment, the system is run to convergence on a single global solution, which then can be tested, validated, utilized or compared as outlined below.

In another embodiment, a global solution is found, and then additional competing models are generated in the neighborhood of the global solution. As will be appreciated by those in the art, this may be done in a wide variety of ways. Assuming convergence on a global solution, any number of sampling techniques may be done. For example, a Monte Carlo search may be done to generate a rank-ordered list of models in the neighborhood of the solution. Starting at the solution, random physiological unit operation changes are made, and a new solution is calculated. If the new model meets the criteria for acceptance, it is used as a starting point for another jump. After a predetermined number of jumps, a rank-ordered list of models is generated. Monte Carlo searching is a sampling technique to explore search space around the global minimum or to find new local minima distant in search space. There are other sampling techniques that can be used, including Boltzman sampling, additional genetic algorithm techniques and simulated annealing. In addition, for all the sampling techniques, the kinds of jumps allowed can be altered (e.g. random jumps to random physiological unit operations, biased jumps (to or away from global solution, for example), etc.) Similarly, for all the sampling techniques, the acceptance criteria of whether a sampling jump is accepted can be altered.

Alternatively, there may not be convergence to a single global model. This may occur in a variety of ways. For example, the iterations may be stopped when a certain finite size set of possible solutions exist; for example, a set of 3-100 possible competing solutions may be desirable. In another example, the members of the solution set (or parent population of psuedochromosomes in GAs) may not change for a finite number of generations (e.g., 10 to 1000 generations), suggesting that a global optimum set has converged. In yet another example, the best (as measured by the fitness function) member(s) of the solution set does (do) not change for several iterations of the algorithm (e.g., 10 to 10000 generations). Similarly, the model may not reach convergence, and will stop with a set of possible solutions. Furthermore, it is possible to generate a set of local minima solutions, using one or more different computational techniques. For example, a GA may be used to generate one or more solutions, and one or more different computational techniques can be used to generate additional set(s) of possible solutions. These can be “pooled” together to form a testable set as outlined below. Once convergence is reached, or a set of solutions is generated, a variety of additional steps are optionally run.

In a preferred embodiment, one or more of the hypothetical mathematical models (preferably one at or near convergence) are compared to one or more additional set(s) of empirical data. For example, a solution to a signaling pathway involved in breast cancer can be used on data from prostrate or lung cancer, etc. This allows comparisons and identifications of similarities and differences within signaling pathways in related systems. Thus, for example, the knowledge that two pathways in two different cancers act either similarly or different is very valuable. This may allow the development of drugs that will act on common pathways (e.g. drugs “generic” to any cancer pathway) or to specific pathways (e.g. drugs that will treat lung cancer but will not effect other tissues). Similarly, it may be very useful to compare models to data generated from untreated and treated cells or animals. That is, there is a variety of data generated from animals or cells that have been treated with drugs or drug candidates as compared to untreated samples. A model generated from either a treated or untreated sample can be compared with the other, to identify either similarities or differences.

In a preferred embodiment, the models are experimentally validated. That is, a model identified either as the global solution or a possible solution can be validated by any number of experimental techniques. In a preferred embodiment one or more of the same parameters are adjusted in competing models “in silico” until competing models predict measureably different outcomes. The same parameter(s) are adjusted in vivo, such as with genetic engineering or metabolic engineering techniques generally known to the art, with the resulting outcome measured and added to the empirical data set. Optionally, the Al algorithm can be rerun against the new “OMICS” data set, or the model that least fits the new result can be dropped from the solution set. This process of prediction and empirical validation can be reiterated until convergence on a single model is reached.

In a preferred embodiment, the Al algorithms of the invention are implemented on any number of different integrated circuits, with preferred embodiments utilizing field-programmable gate arrays (FPGAs) or in application-specific integrated circuits (ASIC) devices to gain additional processing speed. These FPGA or ASIC devices are incorporated as addressable co-processing units in computing systems to free cpu and memory allocation burdens on the computing systems. In addition, these systems may be incorporated into larger systems as outlined below.

The methods of the invention find use in a variety of applications. In particular, the methods of the invention can be used to generate, validate, complete or alter mathematical models of biological function, including disease pathways.

In a preferred embodiment, the systems of the invention are used to elucidate metabolic pathways in any type of prokaryotic or eukaryotic organism (including tissues and cells) or viruses. Suitable prokaryotic cells include, but are not limited to, bacteria such as E. coli, Bacillus species, and the extremophile bacteria such as thermophiles, etc. Suitable eukaryotic cells include, but are not limited to, fungi such as yeast and filamentous fungi, including species of Aspergillus, Trichoderma, and Neurospora; plant cells including those of corn, sorghum, tobacco, canola, soybean, cotton, tomato, potato, alfalfa, sunflower, etc.; and animal cells, including fish, birds and mammals. Suitable fish cells include, but are not limited to, those from species of salmon, trout, tulapia, tuna, carp, flounder, halibut, swordfish, cod and zebrafish. Suitable bird cells include, but are not limited to, those of chickens, ducks, quail, pheasants and turkeys, and other jungle fowl or game birds. Suitable mammalian cells include, but are not limited to, cells from horses, cattle, buffalo, deer, sheep, rabbits, rodents such as mice, rats, hamsters, gerbils, and guinea pigs, minks, goats, pigs, primates, marsupials, marine mammals including dolphins and whales, as well as cell lines, such as human cell lines of any tissue or stem cell type, and stem cells. Preferred systems utilize data from mouse and human cells; this includes the use of data generated in vitro and in vivo, from cells, cell lines, tissues or the whole organism. Accordingly, empirical data may be from any number of different cell types, with human, primate and rodent cells of the following cell types being preferred: tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, COS, etc. See the ATCC cell line catalog, hereby expressly incorporated by reference.

As will be appreciated by those in the art, metabolic pathways within viruses, and within infected host cells, are of growing interest; accordingly, the invention finds use in the elucidation of viral-related pathways (again, either within viruses or within infected host cells); suitable viruses including, but are not limited to, orthomyxoviruses, (e.g. influenza virus), paramyxoviruses (e.g respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus), hepatitis viruses (including A, B and C), herpesviruses (e.g. Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picornaviruses, and the like, and bacteria (including a wide variety of pathogenic and non-pathogenic prokaryotes of interest including Bacillus; Vibrio, e.g. V. cholerae; Escherichia, e.g. enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C. perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. Y. pestis, Pseudomonas, e.g. P. aeruginosa, P. putida; Chlamydia, e.g. C. trachomatis; Bordetella, e.g. B. pertussis; Treponema, e.g. T. palladium; G. lamblia and the like may be used.

In a preferred embodiment, the methods and compositions of the invention find use in target identification. By modeling disease states, the identification of druggable targets and diagnostic biomarkers can be done. That is, knowledge that a particular gene, gene product, or metabolite is specifically involved in a given biological process makes the target a potential candidate for therapeutic intervention. For example, the protein encoded by that gene may be an excellent target for manipulation by small molecule drugs. Similarly, drugs such as antisense and siRNA molecules can be used to alter (in this case, inhibit) the expression of the gene itself. In addition, one can envision therapies involving the delivery of that protein itself to increase its abundance or replace a defective mutant version. Sensitivity analysis of the adjustable parameters of the optimum model can be used to find the most response point in a pathway to affect a therapeutic outcome, hence be used to define the “best” target against which to direct drug development and screening efforts.

Accordingly, in a preferred embodiment, the methods and compositions of the invention find use in the elucidation of models of a variety of disease states, including, but not limited to, cancer (the models may be directed to invasion, metastasis or growth of cancer); disorders associated with: apoptosis; cell death; loss of cell division or decreased cell growth; the regulation and disregulation of angiogenesis; multidrug resistance; the regulation and disregulation of inflammation; membrane depolarization (e.g. in cardiovascular disease, the decrease in arrythmogenic potential of insult); cell swelling; leakage of specific intracellular ions; the regulation and disregulation of ion channels (including potassium and chloride channels); the regulation and disregulation of myosin polymerization/depolymerization (e.g. in cardiovascular disease); calcium cycling; proton pump function; the regulation and disregulation of proteases; the regulation and disregulation of cytokines; obesity; diabetes; cardiovascular disease and plaque formation; osteroporosis; osteoarthritis; arthritis, including rheumatoid arthritis; autoimmune diseases (including lupus, arthritis, multiple sclerosis, diabetes, psoriasis; Chrone's Disease; thyroid disease, etc.)

Once the mathematical model of a particular biochemical pathway incorporating disease-specific parameter changes has been generated, this allows the identification of particular molecules within the disease pathway that serve as targets for drug development. Thus, the methods of the invention may be combined with any number of screening techniques, particularly high-throughput screening (HTS) techniques, that allow the screening of candidate bioactive agents to find drug candidates that affect the target model parameters to bring them back to values consistent with healthy physiologies. Additionally, the model provides information on what components provide the best measureable responses to a drug, or provide markers for other potential side effects of a given therapy.

The choice of suitable targets against which to screen is based on a wide variety of known factors within the art. For example, extracellular or cell surface bound molecules (e.g. cell surface receptors), may be screened with small molecule or antibody libraries. Intracellular molecules may be screened against small molecule libraries, peptides and nucleic acids, etc. Metabolic enzymes that are allosterically regulated with small molecule ligands. Mutant or otherwise defective enzymes or receptor proteins that must be supplemented with effective copies either by injecting the effective protein itself as a therapeutic or using gene therapy to correct or add a non-mutant form of the gene to the genome of the organism.

Accordingly, the present invention includes methods of screening cells with candidate bioactive agents to modulate the activity of target components. “Modulate” in this context can include both agonistic and antagonistic effects (e.g. stimulatory or inhibitory).

The term “candidate bioactive agent” or “candidate drug” as used herein describes any molecule, e.g., protein, small organic molecule, carbohydrates (including polysaccharides), polynucleotide, lipids, synthetic molecules or natural metabolites and their derivatives, etc. Generally a plurality of assay mixtures are run in parallel with different agent concentrations to obtain a differential response to the various concentrations. Typically, one of these concentrations serves as a negative control, i.e., at zero concentration or below the level of detection. In addition, positive controls can be used.

Candidate agents encompass numerous chemical classes, though typically they are organic molecules, preferably small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In a preferred embodiment, the candidate bioactive agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations. Chemical blocking groups or other chemical substituents may also be added.

In a preferred embodiment, the candidate bioactive agents are naturally occurring proteins or fragments of naturally occurring proteins. Thus, for example, cellular extracts containing proteins, or random or directed digests of proteinaceous cellular extracts, may be used. In this way libraries of procaryotic and eukaryotic proteins may be made for screening in the systems described herein. Particularly preferred in this embodiment are libraries of bacterial, fungal, viral, and mammalian proteins, with the latter being preferred, and human proteins being especially preferred.

In a preferred embodiment, the candidate bioactive agents are peptides of from about 5 to about 30 amino acids, with from about 5 to about 20 amino acids being preferred, and from about 7 to about 15 being particularly preferred. The peptides may be digests of naturally occurring proteins as is outlined above, random peptides (including “biased” random peptides). By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, etc., or to purines, etc.

In a preferred embodiment, the candidate bioactive agents are nucleic acids. By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage, et al., Tetrahedron, 49(10):1925 (1993) and references therein; Letsinger, J. Org. Chem., 35:3800 (1970); Sprinzl, et al., Eur. J. Biochem., 81:579 (1977); Letsinger, et al., Nucl. Acids Res., 14:3487 (1986); Sawai, et al., Chem. Lett., 805 (1984), Letsinger, et al., J. Am. Chem. Soc., 110:4470 (1988); and Pauwels, et al., Chemica Scripta, 26:141 (1986)), phosphorothioate (Mag, et al., Nucleic Acids Res., 19:1437 (1991); and U.S. Pat. No. 5,644,048), phosphorodithioate (Briu, et al., J. Am. Chem. Soc., 111:2321 (1989)), O-methylphophoroamidite linkages (see Eckstein, Oligonucleotides and Analogues: A Practical Approach, Oxford University Press), and peptide nucleic acid backbones and linkages (see Egholm, J. Am. Chem. Soc., 114:1895 (1992); Meier, et al., Chem. Int. Ed. Engl., 31:1008 (1992); Nielsen, Nature, 365:566 (1993); Carlsson, et al., Nature, 380:207 (1996), all of which are incorporated by reference)). Other analog nucleic acids include those with positive backbones (Denpcy, et al., Proc. Natl. Acad. Sci. USA, 92:6097 (1995)); non-ionic backbones (U.S. Pat. Nos. 5,386,023; 5,637,684; 5,602,240; 5,216,141; and U.S. Pat. No. 4,469,863; Kiedrowshi, et al., Angew. Chem. Intl. Ed. English, 30:423 (1991); Letsinger, et al., J. Am. Chem. Soc., 110:4470 (1988); Letsinger, et al., Nucleoside & Nucleotide, 13:1597 (1994); Chapters 2 and 3, ASC Symposium Series 580, “Carbohydrate Modifications in Antisense Research”, Ed. Y. S. Sanghui and P. Dan Cook; Mesmaeker, et al., Bioorganic & Medicinal Chem. Lett., 4:395 (1994); Jeffs, et al., J. Biomolecular NMR, 34:17 (1994); Tetrahedron Lett., 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins, et al., Chem. Soc. Rev., (1995) pp. 169-176). Several nucleic acid analogs are described in Rawls, C & E News, Jun. 2, 1997, page 35. All of these references are hereby expressly incorporated by reference. These modifications of the ribose-phosphate backbone may be done to facilitate the addition of additional moieties such as labels, or to increase the stability and half-life of such molecules in physiological environments. In addition, mixtures of naturally occurring nucleic acids and analogs can be made. Alternatively, mixtures of different nucleic acid analogs, and mixtures of naturally occurring nucleic acids and analogs may be made. The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

As described above generally for proteins, nucleic acid candidate bioactive agents may be naturally occurring nucleic acids, random nucleic acids, or “biased” random nucleic acids. For example, digests of procaryotic or eukaryotic genomes may be used as is outlined above for proteins.

In a preferred embodiment, the candidate bioactive agents are organic chemical moieties, a wide variety of which are available in the literature.

In a preferred embodiment, a library of different candidate bioactive agents are used. Preferably, the library should provide a sufficiently structurally diverse population of agents to effect a probabilistically sufficient range of diversity to allow binding to a particular target. Accordingly, an interaction library should be large enough so that at least one of its members will have a structure that gives it affinity for the target. Although it is difficult to gauge the required absolute size of an interaction library, nature provides a hint with the immune response: a diversity of 10⁷-10⁸ different antibodies provides at least one combination with sufficient affinity to interact with most potential antigens faced by an organism. Published in vitro selection techniques have also shown that a library size of 10⁷-10⁸ is sufficient to find structures with affinity for the target. A library of all combinations of a peptide 7 to 20 amino acids in length, such as generally proposed herein, has the potential to code for 20⁷ (10⁹) to 20²⁰. Thus, with libraries of 10⁷ to 10⁸ different molecules the present methods allow a “working” subset of a theoretically complete interaction library for 7 amino acids, and a subset of shapes for the 20²⁰ library. Thus, in a preferred embodiment, at least 10⁶, preferably at least 10⁷, more preferably at least 10⁸ and most preferably at least 10⁹ different sequences are simultaneously analyzed in the subject methods. Preferred methods maximize library size and diversity.

As will be appreciated by those in the art, either in vitro or in vivo (including both ex vivo (e.g. cells) and in vivo (organisms) screening techniques can be used.

In a preferred embodiment, the target molecule is isolated and tested in vitro. In a preferred embodiment, the target protein is isolated, cloned, expressed using well known techniques, and isolated for use in in vitro assays. Target proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the target protein may be purified using a standard anti-library antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the target protein. In some instances no purification will be necessary.

As will be appreciated by those in the art, a wide variety of known in vitro screening systems are known. For example, the target protein or the candidate agent is non-diffusibly bound to an insoluble support having isolated sample receiving areas (e.g. a microtiter plate, an array, etc.). The insoluble supports may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, teflon™, etc. Microtiter plates and arrays are especially convenient because a large number of assays can be carried out simultaneously, using small amounts of reagents and samples. In some cases magnetic beads and the like are included. The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety. Also included in this invention are screening assays wherein solid supports are not used; examples of such are described below.

In a preferred embodiment, the target protein is bound to the support, and a candidate bioactive agent is added to the assay. Alternatively, the candidate agent is bound to the support and the target protein is added. Novel binding agents include specific antibodies, non-natural binding agents identified in screens of chemical libraries, peptide analogs, etc. Of particular interest are screening assays for agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the candidate bioactive agent to the target protein may be done in a number of ways. In a preferred embodiment, the candidate bioactive agent is labelled, and binding determined directly. For example, this may be done by attaching all or a portion of the target protein to a solid support, adding a labeled candidate agent (for example a fluorescent label), washing off excess reagent, and determining whether the label is present on the solid support. Various blocking and washing steps may be utilized as is known in the art.

In a preferred embodiment, cellular assays are done. In this embodiment, the candidate bioactive agents are combined or added to a cell or population of cells comprising the target molecule (which can be either naturally occurring in the cell population (e.g. endogeneous) or recombinately added (e.g. exogeneous to the cell). Suitable cell types for different embodiments are outlined above. The candidate bioactive agent and the cells are combined. As will be appreciated by those in the art, this may accomplished in any number of ways, including adding the candidate agents to the surface of the cells, to the media containing the cells, or to a surface on which the cells are growing or in contact with; adding the agents into the cells, for example by using vectors that will introduce the agents into the cells (i.e. when the agents are nucleic acids or proteins).

In one embodiment, the candidate bioactive agents are either nucleic acids or proteins (proteins in this context includes proteins, oligopeptides, and peptides) that are introduced into the host cells using vectors, including viral vectors. The choice of the vector, preferably a viral vector, will depend on the cell type. When the cells are replicating, retroviral vectors are used as is more fully described below. When the cells are not replicating (i.e. they are arrested in one of the growth phases), other viral vectors may be used, including lentiviral and adenoviral vectors.

In general, the candidate agents are added to the cells (either extracellularly or intracellularly, as outlined above) under reaction conditions that favor agent-target interactions. Generally, this will be physiological conditions. Incubations may be performed at any temperature which facilitates optimal activity, typically between 4 and 40° C. Incubation periods are selected for optimum activity, but may also be optimized to facilitate rapid high through put screening. Typically between 0.1 and 1 hour will be sufficient. Excess reagent is generally removed or washed away.

A variety of other reagents may be included in the assays. These include reagents like salts, neutral proteins, e.g. albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for detection. Washing or rinsing the cells will be done as will be appreciated by those in the art at different times, and may include the use of filtration and centrifugation.

As will be appreciated by those in the art, the cells can be screened in a variety of ways on a variety of bases. In general, the cells are screened for altered phenotypes, correlated with the modulation of the target molecule. By “altered phenotype” or “changed physiology” or other grammatical equivalents herein is meant that the phenotype of the cell is altered in some way, preferably in some detectable and/or measurable way. As will be appreciated in the art, a strength of the present invention is the wide variety of cell types and potential phenotypic changes which may be tested using the present methods. Accordingly, any phenotypic change which may be observed, detected, or measured may be the basis of the screening methods herein. Suitable phenotypic changes include, but are not limited to: gross physical changes such as changes in cell morphology, cell growth, cell viability, adhesion to substrates or other cells, and cellular density; changes in the expression of one or more RNAs, proteins, lipids, hormones, cytokines, or other molecules; changes in the equilibrium state (i.e. half-life) or one or more RNAs, proteins, lipids, hormones, cytokines, or other molecules; changes in the localization of one or more RNAs, proteins, lipids, hormones, cytokines, or other molecules; changes in the bioactivity or specific activity of one or more RNAs, proteins, lipids, hormones, cytokines, receptors, or other molecules; changes in the secretion of ions, cytokines, hormones, growth factors, or other molecules; alterations in cellular membrane potentials, polarization, integrity or transport; changes in infectivity, susceptability, latency, adhesion, and uptake of viruses and bacterial pathogens; etc. By “capable of altering the phenotype” herein is meant that the bioactive agent can change the phenotype of the cell in some detectable and/or measurable way.

The altered phenotype may be detected in a wide variety of ways, and will generally depend and correspond to the phenotype that is being changed. Generally, the changed phenotype is detected using, for example: microscopic analysis of cell morphology; standard cell viability assays, including both increased cell death and increased cell viability, for example, cells that are now resistant to cell death via virus, bacteria, or bacterial or synthetic toxins; standard labeling assays such as fluorometric indicator assays for the presence or level of a particular cell or molecule, including FACS or other dye staining techniques; biochemical detection of the expression of target compounds after killing the cells; mass spectroscopy; capillary electrophoresis; As will be appreciated by those in the art, screening is frequently done on the basis of the incorporation of a label in the screening system. That is, in one embodiment, some component of the assay system is labeled. By “labeled” herein is meant that nucleic acids, proteins and antibodies of the invention have at least one element, isotope or chemical compound attached to enable the detection of nucleic acids, proteins and antibodies of the invention. In general, labels fall into three classes: a) isotopic labels, which may be radioactive or heavy isotopes; b) immune labels, which may be antibodies or antigens; and c) colored or fluorescent dyes, although labels such as enzymes (alkaline phosphotase and HRP), beads (e.g. magnetic beads, etc.) can also be used. The labels may be incorporated into the compound at any position.

In such empirical screening assays the result is limited to the measured parameter or outcome. By coupling said screening methods (supra) with physiologically-relevant models, it is possible to interpret the actual point of interaction in the biochemical pathway and what model parameter (e.g., enzyme rate constant, Michaelis constant, equilibrium constant or competitive equilibrium is affected by the bioactive compound. In this way differences in the mechanism of action of compounds with substantially the same observable empirical activity can be discerned.

In a preferred embodiment, once a cell with an altered phenotype is detected, the cell is isolated from the plurality which do not have altered phenotypes. This may be done in any number of ways, as is known in the art, and will in some instances depend on the assay or screen. Suitable isolation techniques include, but are not limited to, FACS, lysis selection using complement, cell cloning, scanning by Fluorimager, expression of a “survival” protein, induced expression of a cell surface protein or other molecule that can be rendered fluorescent or taggable for physical isolation; expression of an enzyme that changes a non-fluorescent molecule to a fluorescent one; overgrowth against a background of no or slow growth; death of cells and isolation of DNA or other cell vitality indicator dyes, etc.

In a preferred embodiment, the bioactive agent is isolated from the positive cell. This may be done in a number of ways as is known in the art. Once rescued, the sequence of the bioactive agent and/or bioactive nucleic acid is determined. This information can then be used in a number of ways. In a preferred embodiment, the bioactive agent is resynthesized and reintroduced into the target cells, to verify the effect. This may be done as in known in the art.

In a preferred embodiment, the bioactive agent is used to pull out target molecules. For example, as outlined herein, if the target molecules are proteins, the use of epitope tags or purification sequences can allow the purification of primary target molecules via biochemical means (co-immunoprecipitation, affinity columns, etc.). Alternatively, the peptide, when expressed in bacteria and purified, can be used as a probe against a bacterial cDNA expression library made from mRNA of the target cell type. Or, peptides can be used as “bait” in either yeast or mammalian two or three hybrid systems. Such interaction cloning approaches have been very useful to isolate DNA-binding proteins and other interacting protein components. The peptide(s) can be combined with other pharmacologic activators to study the epistatic relationships of signal transduction pathways in question.

The screening methods of the present invention may be useful to screen a large number of cell types under a wide variety of conditions. Generally, the host cells are cells that are involved in disease states, and they are tested or screened under conditions that normally result in undesirable consequences on the cells. When a suitable bioactive agent is found, the undesirable effect may be reduced or eliminated. Alternatively, normally desirable consequences may be reduced or eliminated, with an eye towards elucidating the cellular mechanisms associated with the disease state or signalling pathway.

The assays of the invention can utilize robotic systems. In a preferred embodiment, the devices of the invention comprise liquid handling components, including components for loading and unloading fluids at each station or sets of stations. The liquid handling systems can include robotic systems comprising any number of components. In addition, any or all of the steps outlined herein may be automated; thus, for example, the systems may be completely or partially automated.

As will be appreciated by those in the art, there are a wide variety of components which can be used, including, but not limited to, one or more robotic arms; plate handlers for the positioning of microplates; holders with cartridges and/or caps; automated lid or cap handlers to remove and replace lids for wells on non-cross contamination plates; tip assemblies for sample distribution with disposable tips; washable tip assemblies for sample distribution; 96 well loading blocks; cooled reagent racks; microtitler plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In a preferred embodiment, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In a preferred embodiment, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station.

In a preferred embodiment, thermocycler and thermoregulating systems are used for stabilizing the temperature of the heat exchangers such as controlled blocks or platforms to provide accurate temperature control of incubating samples from 4BC to 100 BC; this is in addition to or in place of the station thermocontrollers.

In a preferred embodiment, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

These instruments can fit in a sterile laminar flow or fume hood, or are enclosed, self-contained systems, for cell culture growth and transformation in multi-well plates or tubes and for hazardous operations. The living cells will be grown under controlled growth conditions, with controls for temperature, humidity, and gas for time series of the live cell assays. Automated transformation of cells and automated colony pickers will facilitate rapid screening of desired cells.

Flow cytometry or capillary electrophoresis formats can be used for individual capture of magnetic and other beads, particles, cells, and organisms.

The flexible hardware and software allow instrument adaptability for multiple applications. The software program modules allow creation, modification, and running of methods. The system diagnostic modules allow instrument alignment, correct connections, and motor operations. The customized tools, labware, and liquid, particle, cell and organism transfer patterns allow different applications to be performed. The database allows method and parameter storage. Robotic and computer interfaces allow communication between instruments.

In a preferred embodiment, the robotic apparatus includes a central processing unit which communicates with a memory and a set of input/output devices (e.g., keyboard, mouse, monitor, printer, etc.) through a bus. Again, as outlined below, this may be in addition to or in place of the CPU for the multiplexing devices of the invention. The general interaction between a central processing unit, a memory, input/output devices, and a bus is known in the art. Thus, a variety of different procedures, depending on the experiments to be run, are stored in the CPU memory. As will be appreciated by those in the art, Mathematica and MathCad programs and compatible programs can be used.

These robotic fluid handling systems can utilize any number of different reagents, including buffers, reagents, samples, washes, assay components, etc.

While the above discussion has focused on biological applications, as outlined herein, other systems can be evaluated and mathematical models generated. For example, in one embodiment, the unit operations are traffic related. The effects that can be modeled include this season/holidays variations, time of day, presence or absence of construction, the presence or absence of entertainment events such as sporting events or concerts, etc. Unit operations may include: traffic signals, intersections crosswalks or ramps, tunnels, bridges, highways, streets, parking structures, etc. The objects upon which the unit operations act may include, pedestrians, automobiles, trucks, trains, bicycles, etc.

In another embodiment, the models are weather related, including models for seasons, temperature wind speed, precipitation, global warming or climate change, etc. Unit operations may include, thermal convection in air and water, solar radiation and absorption, evaporation, condensation, forced convection (the effects of wind and water), point sources of heating or cooling (icebergs, glaciers, volcanos, human activities).

In another embodiment, the models are financial, economic, or market analysis related, including, models for stock pricing, capital movement, inflation, the pricing or placement of goods and services, advertising effectiveness, etc. The unit operations may include, banks, government monetary and fiscal policies, manufacturing operations, transportation, construction, wholesale and retail spending habits, etc.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes.

All references, including those listed below as well as cited herein are incorporated by reference in their entirety:

1. Pauling, L., “The oxygen equilibrium of hemoglobin and its structural interpretation,” Science, 81:421 (1935); Pauling, L., “The oxygen equilibrium of hemoglobin and its structural interpretation,” Proc. Natl. Acad. Sci. (USA), 21:186-191 (1935).

2. Watson, J. D. and F. H. C. Crick, “A structure for deoxyribose nucleic acid,” Nature, 171:737 (1953).

3. Sanger, F., “The free amino groups of insulin,” Biochem. J., 39:507-515 (1945); Sanger, F. and H. Tuppy, “The amino-acid sequence in the phenylalanine chain of insulin: I. The identification of lower peptides from partial hydrosylates,” Biochem. J., 49:463-481 (1951); Sanger, F. and H. Tuppy, “The amino-acid sequence in the phenylalanyl chain of insulin: II. The investigation of peptides from enzymic hydrolysates,” Biochem. J., 49:481-490 (1951); Sanger, F. and E. O. P. Thompson, “The amino-acid sequence in the glycyl chain of insulin,” Biochem. J. 53: 353-374 (1953); Sanger, F., E. O. P. Thompson, and R. Kitai, “The amide groups of insulin,” Biochem. J., 59:509-518 (1955).

4. Jensen, H. and E. A. Evans, “Studies on crystalline insulin: XVIII. The nature of the free amino groups in insulin and the isolation of phenylalanine and proline from crystalline insulin,” J. Biol. Chem., 108:1-9 (1935).

5. Holley, R. W., J. Apgar, G. A. Everett, J. T. Madison, M. Marguisee, S. H. Merrill, J. R. Penswick, and A. Zamir, “Structure of a ribonucleic acid,” Science, 147:1462-1465 (1965).

6. U.S. Census Bureau, International Programs Center, World Population Estimaate, http://www.census.gov/main/www/popclock.html (April, 29, 2002).

7. George Post, speech at the BIO 2000 Conference, Boston, Mass., (Feb, 2000).

8. Uetz, P. et al., “A comprehesive analysis of protein-protein interactions in Saccharomyces cerevisiase,” Nature, 403:623-627 (2000).

9. van't Veer, L. J. et al., “Gene expression profiling predits clinical outcome of breast cancer,” Nature, 415:530-536 (2002).

10. Fiehn, O., “Metabolomics: Future prospects and challenges,” presented at the BIO 2002 conference, Toronto, Ontario, Canada (Jun. 10, 2002).

11. Drew, J., “Drug discovery: a historical perspective,” Science, 287:1960-1964 (2000).

12. Drew, J., “Drug discovery: a historical perspective,” Science, 287:1960-1964 (2000). Ref for # of chemical compounds.

13. Ernst and Young report. Casdin, Jeff, CEO Casdin Capital Partners, speech given at the CHI Genomics Opportunities Conference (Feb 14-15, 1998); Drew, J., “Drug discovery: a historical perspective,” Science, 287:1960-1964 (2000).

14. Lederberg, J. A. and A. T. McCray, “Ome Sweet 'Omics—A genealogical treasury of words,” The Scientist, 15:8 (2001).

15. Anderson, L. and J. Seilhamer, Electrophoresis, 18:533 (1997).

16. Gygi, S. P., Rochon, Y., Franza, B. R., and R. Aebersold, “Correlation between protein and mRNA abundance in yeast,” Mol. Cell. Biol., 19:1720-1730 (1999).

17. Aebersold, R. H., et al., “Rapid quantitative analysis of proteins or protein function in complex mixtures,” WO 00/11208 (Mar. 2, 2000).

18. Schneider, L. V. et al., WO 01/49951 (in press); Hall, M. P. et al., “Inverted mass ladder sequencing (IMLS™): A method for sequencing intact proteins by in-source fragmentation using novel ‘mass defect’ labels,” paper presented at the Amer. Soc. Mass Spectro. 50th Annual Meeting, Orlando, Fla. (June, 2002).

19. Gonzalez, B., Piotto, M., and G. Huber, “Method and device for analyzing the intracellular chemical state of living cells by nuclear magnetic resonance,” WO 02/31523A1 (Apr. 18, 2002).

20. Schneider, L. V. et al., “Polypeptide fingerprinting methods, metabolic profiling, and bioinformatics database,” WO 00/63683 (Oct. 26, 2000).

21. Murray, J. D., An Introduction to Mathematical Biology, 3rd ed. (Springer-Verlag, 2002).

22. International Society for Computational Biology (www.iscb.org).

23. Price Waterhouse Coopers, “Pharma 2005 Silicon Rally, the Race to e-R&D” (1999).

24. Brown A. J., J. Chem Soc. (Trans.) 61:369-385 (1982).

25. Henri, V., C. r. hebd. Acad. Sci. Paris, 135:916-919 (1902); Henri, V. (1903) Lois Generales de I'Action des Diastases, (Hermann, Paris, 1903).

26. Michaelis L. and Menten, M. L., Biochem. Z., 49:333-369 (1913).

27. Pauling, L., “The oxygen equilibrium of hemoglobin and its structural interpretation,” Proc. Natl. Acad. Sci. (USA), 21:186-191 (1935).

28. Monod, J., “The growth of bacterial cultures,” Ann. Rev. Microbiol., 3:371 (1949).

29. Monod, J., Wyman, J., and F. Jacob, J. Mol. Biol., 12:88 (1965).

30. Chock, P. B. and E. R. Stadtman, Proc. Natl. Acad. Sci. (USA), 74:2766 (1977); Stadtman, E. R. and P. B. Chock, Proc. Natl. Acad. Sci. (USA), 74:2761 (1977); Stadtman E. R. and P. B. Chock, Curr. Top. Cell. Regul., 13:53 (1978).

31. Schneider, L. V., “Metabolic uncoupling in Escherichia coli during phosphate-limited growth,” PhD. Dissertation, Vol. 2, Dept. of Chem. Eng. (Princeton Univ, Princeton, N.J., 1997).

32. Schneider, L. V. and W. F. Stahl, “G-Protein coupled receptor (GPCR) based biosensors and sense replication systems,” PCT Patent Appl. No. WO 00/70343 (Nov. 23, 2000).

33. Beset, D. H., Object-oriented implementation of numerical methods: An introduction with Java and Smalltalk (Morgan Kaufmann, San Francisco, 2001).

34. McNeil, M., personal communication, Dept. of Chem. Eng., San Jose State Univ. (April, 2002).

35. Gear, C. W., Numerical Initial Value Problems in Differential Equations (Prentice Hall: Englewood Cliffs, N.J., 1970).

36. Hairer, E. and G. Wanner, Solving Ordinary Differential Equations II: Stiff and Differential-Algebraic Problems (Springer-Verlag, Berlin, 1991).

37. Sandu, A. et al., “Benchmarking stiff ODE solvers for atmospheric chemistry problems I: Implicit vs. explicit,” Atmospheric Environment, 31:3151-3166 (1997); Sandu, A. et al., “Benchmarking stiff ODE solvers for atmospheric chemistry problems II: Rosenbrock methods,” Atmospheric Environment, 31:3459-3472 (1997).

38. Macey, R., Oster, G., Zahnley, T., Berkeley Madonna User's Guide (University of California, Berkeley, Dept. of Mol. and Cell. Biol., 2000).

39. Lehninger, A. L., Biochemistry (Worth, NY, 1975); Stryer, L., Biochemistry (W. H. Freeman, NY, 1981).

40. Michal, G. ed., Biochemical Pathways, 3rd ed., http://www.roche-applied-science.com/prodinfo_fst.htm?/techserv/metmap.htm (Roche Molecular Biochemicals, 2002). Karp, P. D. et al., “The EcoCyc database,” Nuc. Acids Res., 30:56-58 (2002); http://www.ecocyc.org/.

41. McAdams, H. H., Arkin, A. P., L. Shapiro, “System and method for simulating operation of biochemical systems,” U.S. Pat. No. 5,914,891 (Jun. 22, 1999).

42. Paterson, T. S. and A. L. Bangs, “Method of providing access to object parameters within a simulations model,” US 6069629 (May 30, 2000); Paterson, T. S., Holtzmann, S., and A. L. Bangs, “Method of generating a display for a dynamic simulation model utilizing node and link representations,” U.S. Pat. No. 6,051,029 (Apr. 18, 2000); Paterson, T. S. and A. L. Bangs, “Method of managing objects and parameter values within a simulation model,” U.S. Pat. No. 6,078,739 (Jun. 20, 2000).

43. Winslow, R., Rounds, D., and D. Scollan, “Computational system and method for modeling the heart,” U.S. Pat. No. 5,947,899 (Sep. 7, 1999); Winslow, R. L. et al., “System and method for modeling genetic, biochemical, biophysical, and anatomical information,” WO 00/65523 (Nov. 2, 2000); Colatsky, T. J. et al., “Computational system for modelling protein expression in an organ,” WO 01/98935 (Dec. 27, 2001); Rice, J. J. et al., “Method and system for modeling biological systems, WO 02/05205 A2 (Jan. 17, 2002); Jim, K.-C. et al., “System for modeling biological pathways,” WO 02/44992 A2 (Jun. 6, 2002).

44. Aebersold, R., Biemann Award Lecture, Amer. Soc. Mass Spectro., 50th Ann. Meeting, Orlando, Fla. (June, 2002).

45. Rabaey, J. M. and A. P. Chandrakasan, Digital Integrated Circuits, 2nd ed. (Prentice-Hall, NY, 2002); Weste, N. H. E. and K. Eshraghian, Principles of CMOS VLSI Design: A systems perspective, 2nd ed. (Addison Wesley Longman, NY, 2000).

46. Department of Electrical Engineering and Computer Science, EECS Main Office, 231 Cory Hall #1770, University of California, Berkeley, Calif., http://bwrc.eecs.berkeley.edu/Classes/IcBook/SPICE.

47. Shackleford, B. et al., “Synthesis of minimum-cost multilevel logic networks via genetic algorithm,” IEICE Trans. Fundamentals, E83-A:2528-2537 (2000).

48. Brayton, R. et al., “MIS: A multiple-level logic optimization system,” IEEE Trans. Comput.-Aided Des. Integrated Circuits & Syst., CAD-6:1062-1081 (1987).

49. Devadas, S. et al., “Boolean decomposition in multilevel logic optimization,” IEEE J. Solid State Circuits, 24:399-408 (1989).

50. Rubin, S. M., Computer Aids for VLSI Design, (Static Free Software, Portola Valley, Calif., 1994).

51. McAdams, H. H., Arkin, A. P., and L. Shapiro, “System and method for simulating operation of biochemical systems,” U.S. Pat. No. 5,914,891 (Jun. 22, 1999).

52. McCabe, W. L. and J. C. Smith, Unit Operations in Chemical Engineering, 3rd ed. (McGraw-Hill, NY, 1976).

53. Aspen Plus (@AspenTechnology, Inc., Cambridge, Mass.); PES™ & SciSci@ (Simulation Sciences, Inc., Brea, Calif.).

54. Bailey, J. E. and D. F. Ollis, Biochemical Engineering Fundamentals (McGraw-Hill, NY, 1977).

54. Coughanowr, D. R. and L. B. Koppel, Process Systems Analysis and Control (McGraw-Hill, NY, 1965); Shinskey, F. G., Process-Control Systems (McGraw-Hill, NY, 1979); Ray, W. H., Advanced Process Control (McGraw-Hill, NY, 1981).

55. Koshland, D. E., A. Goldbeter and J. B. Stock, Science, 217:220 (1982).

56. Schneider, L. V., “Metabolic uncoupling in Escherichia coli during phosphate-limited growth,” PhD Dissertation, Dept. of Chem. Eng. (Princeton Univ., Princeton, N.J., 1997).

57. Bowling, K., VP Software Engineering, Entelos, Inc., presentation at the Bay Area Bioinformatics Group, Stanford University (April, 2002).

58. Schneider, L. V., “Metabolic uncoupling in Escherichia coli during phosphate-limited growth,” PhD. Dissertation, Vol. 2, Dept. of Chem. Eng. (Princeton Univ, Princeton, N.J., 1997).

59. Schneider, L. V. and W. F. Stahl, “G-Protein coupled receptor (GPCR) based biosensors and sense replication systems,” PCT Patent Appl. No. WO 00/70343 (Nov. 23, 2000).

60. Schneider, L. V. et al., “Polypeptide fingerprinting methods, metabolic profiling, and bioinformatics database,” WO 00/63683 (Oct. 26, 2000).

61. DeMasi, J. A., “New drug innovation and pharmaceutical industry structure,” Tufts Center for the Study of Drug Development, Tufts University (Oct. 6, 2000)

62. Price Waterhouse Coopers, “Pharma 2005 Silicon Rally, the Race to e-R&D” (1999).

63. DeMasi, J. A., “New drug innovation and pharmaceutical industry structure,” Tufts Center for the Study of Drug Development, Tufts University (Oct. 6, 2000)

64. Boston Consulting Group (1993), as reported in: DeMasi, J. A., “New drug innovation and pharmaceutical industry structure,” Tufts Center for the Study of Drug Development, Tufts University (Oct. 6, 2000).

65. DataEdge, LLC, as reported in: DeMasi, J. A., “New drug innovation and pharmaceutical industry structure,” Tufts Center for the Study of Drug Development, Tufts University (Oct. 6, 2000)

66. Casdin, J., keynote speech, CHI Genomics Opportunities Conference, San Francisco, Calif. (Feb 14-15, 1998).

67. Staff Reporter, “Beyond the Behemoths,” The Economist, pg 346 (Feb. 21, 1998).

68. Edwards, M. G., presented paper, UCSF and Foley & Lardner Bioscience Capital Access Conference, San Francisco, Calif. (Sept., 20-21,2001).

69. Edwards, M. G., presented paper, UCSF and Foley & Lardner Bioscience Capital Access Conference, San Francisco, Calif. (Sept., 20-21,2001).

70. Casdin, J., keynote speech, CHI Genomics Opportunities Conference, San Francisco, Calif. (Feb 14-15, 1998); Owen, C., “In vitro diagnostics,” Private Consulting Study, (Owen, Pambianchi & Assoc., 1997); Gentile, F., “Genomics”, SRI International Techmonitoring Report, (SRI International, San Francisco, Calif., 1998).

71. DeMasi, J. A., “New drug innovation and pharmaceutical industry structure,” Tufts Center for the Study of Drug Development, Tufts University (Oct. 6, 2000).

72. Committee on Ways and Means, U.S. House of Representatives, 105th Congress, WMCP-105-2 (U.S. Government Printing Office, Washington, D.C., 1997).

73. Committee on Ways and Means, U.S. House of Representatives, 105th Congress, WMCP-105-2 (U.S. Government Printing Office, Washington, D.C., 1997).

74. Frazen, F. et al., Electrophoresis, 18:582 (1997).

75. Parekh, R. B., presented paper, IBC Proteomics Conference, Coronado, Calif. (Jun. 11-12,1998); Steiner, S., presented paper, IBC Proteomics Conference, Coronado, Calif. (June 11-12,1998); Anderson, L., presented paper, IBC Proteomics Conference, Coronado, Calif. (Jun. 11-12,1998).

76. Anderson, L., presented paper, IBC Proteomics Conference, Coronado, Calif. (Jun. 11-12,1998).

77. van't Veer, L. J. et al., “Gene expression profiling predicts clinical outcome of breast cancer,” Nature 415:530-536 (2002).

78. Petricoin, E. F. et al., “Use of proteomic patterns in serum to identify ovarian cancer,” Lancet, 359:572-577 (2002).

### EXAMPLES

### Example 1

### Unimolecular Chemical Equilibria

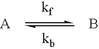

The rate of change in the component A concentration is given by:

$$\frac{ⅆ B [ t ]}{ⅆ t} = {k}_{f} A [ t ] - {k}_{b} B [ t ] \qquad ( 1 )$$

The concentrations of each component can be represented in terms of a deviation (ΔA[t] and ΔB[t]) from a control or steady-state condition (A_{SS} and B_{SS}).

$$\begin{matrix}A [ t ] = {A}_{ss} + Δ A [ t ] & ( 2 ) \\ B [ t ] = {B}_{ss} + Δ B [ t ] & ( 3 ) \\ and \text{:} \frac{ⅆ B [ t ]}{ⅆ t} = \frac{ⅆ {B}_{ss}}{ⅆ t} + \frac{ⅆ Δ B [ t ]}{ⅆ t} & ( 4 ) \\ but , \frac{ⅆ {B}_{ss}}{ⅆ t} = {k}_{f} {A}_{ss} - {k}_{b} {B}_{ss} & ( 5 )\end{matrix}$$

On substitution into equation 1 we get:

$$\frac{ⅆ Δ B [ t ]}{ⅆ t} = {k}_{f} Δ A [ t ] - {k}_{b} Δ B [ t ] \qquad ( 6 )$$

Taking the Laplace transform we get:

sΔB[s]=k_{f}ΔA[s]−k_{b}ΔB[s] (7)

which, solving for ΔA[s] and letting K_{eq}=k_{f}/k_{b} yields

$$Δ B [ s ] = ( \frac{G}{τ s + 1} ) Δ A [ s ]$$

where, the gain in the system is defined by:

G=K_{eq} (9)

and the first order time constant is defined by:

τ=K_{eq}k_{f}⁻¹ (10)

Taking the inverse Laplace transform, we get the solution of the difference equation in the time domain as,

$$\frac{Δ B [ t ]}{Δ A [ t ]} = G {ⅇ}^{- t / τ} \qquad ( 11 )$$

### Example 2

### Bimolecular Chemical Equilibria

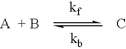

The rate of conversion of substrate concentrations (A and B) to product concentration (C) is given by:

$$\frac{ⅆ C [ t ]}{ⅆ t} = {k}_{f} A [ t ] B [ t ] - {k}_{b} C [ t ] \qquad ( 2.1 )$$

However, the variation in the concentrations of each species can be related to a control or steady-state

[ss] concentrations by:

$$\begin{matrix}A [ t ] = {A}_{ss} + Δ A [ t ] & ( 2.2 ) \\ B [ t ] = {B}_{ss} + Δ B [ t ] & ( 2.3 ) \\ C [ t ] = {C}_{ss} + Δ C [ t ] & ( 2.4 ) \\ and , \frac{ⅆ C ( t )}{ⅆ t} = \frac{ⅆ {C}_{ss}}{ⅆ t} + \frac{ⅆ Δ C [ t ]}{ⅆ t} & ( 2.5 ) \\ but , \frac{ⅆ {C}_{ss}}{ⅆ t} = {k}_{f} {A}_{ss} {B}_{SS} - {k}_{b} {C}_{ss} & ( 2.5 )\end{matrix}$$

Therefore, on substitution into equation 2.1, we get:

$$\frac{ⅆ Δ C [ t ]}{ⅆ t} = {k}_{f} ( {B}_{ss} Δ A [ t ] + {A}_{ss} Δ B [ t ] ) - {k}_{b} Δ C [ t ] + {k}_{f} Δ A [ t ] Δ B [ t ] \qquad ( 2.6 )$$

but to a first approximation, where ΔA[t] and ΔB[t] are both small, the second order term (ΔA[t]ΔB[t]) can

be neglected, yielding the linear approximation:

$$\frac{ⅆ Δ C [ t ]}{ⅆ t} ≅ {k}_{f} ( {B}_{ss} Δ A [ t ] + {A}_{ss} Δ B [ t ] ) - {k}_{b} Δ C [ t ] \qquad ( 2.7 )$$

Taking the Laplace transform and solving for ΔC[s], and letting K_{eq}=k_{f}/k_{b}, we get:

$$Δ C [ s ] = ( \frac{{G}_{1}}{τ s + 1} ) Δ A [ s ] + ( \frac{{G}_{2}}{τ s + 1} ) Δ B [ s ] \qquad ( 2.8 )$$

where,

G₁=K_{eq}Bₛₛ (2.9)

G₂=K_{eq}Aₛₛ (2.10)=

τ=K_{eq}k_{f}⁻¹ (2.11)

Taking the inverse Laplace transform yields the solution in the time domain:

ΔC[t]=(G₁ΔA[t]+G₂ΔB[t])e^{-γ}^{ₜ} (2.12)

### Example 3

### Enzyme-Mediated Equilibrium

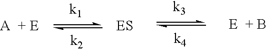

The rate of production of the product B is related to the concentrations of the other components by:

$$\frac{ⅆ B [ t ]}{ⅆ t} = {k}_{3} E S [ t ] - {k}_{4} E [ t ] B [ t ] \qquad ( 3.1 )$$

where the standard pseudosteady-state approximation for the formation of the enzyme complex is applied:

$$\begin{matrix}\frac{ⅆ ES [ t ]}{ⅆ t} = 0 = {k}_{1} A [ t ] E [ t ] + {k}_{4} B [ t ] E [ t ] - ( {k}_{2} + {k}_{3} ) ES [ t ] & ( 3.2 ) \\ and ES [ t ] = \frac{{k}_{1} A [ t ] E [ t ] + {k}_{4} B [ t ] E [ t ]}{( {k}_{2} + {k}_{3} )} & ( 3.3 )\end{matrix}$$

But, from the material balance for the enzyme:

E[t]=E_{T}[t]−ES[t] (3.4)

On substitution into equation 3.1,

$$\frac{ⅆ B [ t ]}{ⅆ t} = \frac{( {k}_{1} {k}_{3} A [ t ] - {k}_{2} {k}_{4} B [ t ] ) {E}_{T} [ t ]}{{k}_{2} + {k}_{3} + {k}_{1} A [ t ] + {k}_{4} B [ t ]} \qquad ( 3.5 )$$

However, the variation in the concentrations of each species can be related to a control or steady-state

[ss] concentrations by:

A[t]=Aₛₛ+ΔA[t] (3.6)

B[t]=Bₛₛ+ΔB[t] (3.7)

E_{T}[t]=Eₛₛ+ΔE[t]tm (3.8)

$$\begin{matrix}and , \frac{ⅆ B [ t ]}{ⅆ t} = \frac{ⅆ {B}_{ss}}{ⅆ t} + \frac{ⅆ Δ B [ t ]}{ⅆ t} & ( 3.9 ) \\ but , \frac{ⅆ {B}_{ss}}{ⅆ t} = \frac{( {k}_{1} {k}_{3} {A}_{ss} - {k}_{2} {k}_{4} {B}_{ss} ) {E}_{T} [ ss ]}{{k}_{2} + {k}_{3} + {k}_{1} {A}_{ss} + {k}_{4} {B}_{ss}} & ( 3.10 )\end{matrix}$$

On substitution and solving for ΔB′[t] and neglecting the second order deviation terms, we get the difference equation:

$$\frac{ⅆ Δ B [ t ]}{ⅆ t} = \frac{( {k}_{1} {k}_{3} Δ A [ t ] - {k}_{2} {k}_{4} Δ B [ t ] ) {E}_{ss}}{{k}_{2} + {k}_{3} + {k}_{1} {A}_{ss} + {k}_{4} {B}_{ss}} + \frac{( {k}_{1} {k}_{3} {A}_{ss} - {k}_{2} {k}_{4} {B}_{ss} ) Δ E [ t ]}{{k}_{2} + {k}_{3} + {k}_{1} {A}_{ss} + {k}_{4} {B}_{ss}} \qquad ( 3.11 )$$

Taking the Laplace transform and solving for ΔB[s], we get:

$$\begin{matrix}Δ B [ s ] = ( \frac{{G}_{1}}{τ s + 1} ) Δ A [ s ] + ( \frac{{G}_{2}}{τ s + 1} ) Δ E [ s ] & ( 3.12 ) \\ where , {G}_{1} = \frac{{k}_{1} {k}_{3}}{{k}_{2} {k}_{4}} & ( 3.13 ) \\ {G}_{2} = \frac{{k}_{1} {k}_{3} {A}_{ss} - {k}_{2} {k}_{4} {B}_{ss}}{{k}_{2} {k}_{4} {E}_{ss}} & ( 3.14 ) \\ and τ = \frac{( {k}_{2} + {k}_{3} + {k}_{1} {A}_{ss} + {k}_{4} {B}_{ss} )}{{k}_{2} {k}_{4} {E}_{ss}} & ( 3.15 )\end{matrix}$$

Taking the inverse Laplace transform we get the solution to the difference equation in the time domain:

ΔB[t]=(G₁ΔA[t]+G₂ΔE[t])e^{-γ}^{_{T}} (3.16)

### Example 4

### Michaelis-Menton kinetics

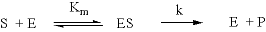

The derivation of the classic Michaelis-Menton equation can be found in any biochemistry text book and yields the rate of product (P) production as:

$$\frac{ⅆ P [ t ]}{ⅆ t} = \frac{k {E}_{T} [ t ] S [ t ]}{{K}_{m} + S [ t ]} \qquad ( 4.1 )$$

Defining the deviation of each concentration from a control or steady-state value as:

S[t]=Sₛₛ+ΔS[t] (4.2)

E[t]=Eₛₛ+ΔE[t] (4.3)

P[t]=Pₛₛ+ΔP[t] (4.4)

$$\begin{matrix}and , \frac{ⅆ P [ t ]}{ⅆ t} = \frac{ⅆ {P}_{ss}}{ⅆ t} + \frac{ⅆ Δ P [ t ]}{ⅆ t} & ( 4.5 ) \\ but , \frac{ⅆ {P}_{ss}}{ⅆ t} = \frac{{kE}_{ss} {S}_{ss}}{{K}_{m} + {S}_{ss}} & ( 4.6 )\end{matrix}$$

On substitution, therefore, we can solve for the difference equation:

$$\frac{ⅆ Δ P [ t ]}{ⅆ t} = \frac{k ( {E}_{ss} + Δ E [ t ] ) ( {S}_{ss} + Δ S [ t ] )}{{K}_{m} + {S}_{ss} + Δ S [ t ]} - \frac{{kE}_{ss} {S}_{ss}}{{K}_{m} + {S}_{ss}} \qquad ( 4.7 )$$

If we neglect small deviations from the control substrate concentration (Sss) in the denominator,

equation 4.7 can be linearized with the approximate relationship:

$$\frac{ⅆ Δ P [ t ]}{ⅆ t} \approx \frac{{kE}_{ss} Δ S [ t ] + Δ E [ t ] {S}_{ss} + Δ E [ t ] Δ S [ t ]}{{K}_{m} + {S}_{ss}} \qquad ( 4.8 )$$

Finally, we can neglect the second order deviation (ΔE[t]ΔS[t]) and get:

$$\frac{ⅆ Δ P [ t ]}{ⅆ t} \approx \frac{{kE}_{ss} Δ S [ t ] + Δ E [ t ] {S}_{ss}}{{K}_{m} + {S}_{ss}} \qquad ( 4.9 )$$

Taking the Laplace transform and solving for P[s], we get:

$$\begin{matrix}Δ P [ s ] = \frac{{G}_{1}}{s} Δ S [ s ] + \frac{{G}_{2}}{s} Δ E [ s ] & ( 4.10 ) \\ where , {G}_{1} = \frac{k {E}_{ss}}{{K}_{m} + {S}_{ss}} & ( 4.11 ) \\ and , {G}_{2} = \frac{k {S}_{ss}}{{K}_{m} + {S}_{ss}} & ( 4.12 )\end{matrix}$$

Taking the inverse Laplace transform yields the solution to the difference equation in the time domain as:

ΔP[t]=G₁ΔS[t]+G₂ΔE[t] (4.13)

### Example 5

### Biomolecular Enzyme Reaction

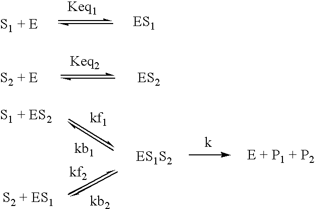

The rate of product production (either P₁ or P₂) is given by:

$$\frac{ⅆ P [ t ]}{ⅆ t} = k {ES}_{1} {S}_{2} [ t ] \qquad ( 5.1 )$$

Assuming the rate of substrate binding is not limiting then the formation of the intermediates (ES1 and ES2) can be described by equilibrium relationships:

ES₁[t]=Keq₁E[t]S₁[t] (5.2)

ES₂[t]=Keq₂E[t]S₂[t] (5.3)

and the total enzyme concentration (ET[t]) can be obtained from the material balance:

E_{T}[t]=E[t]+ES₁[t]+ES₂[t]+ES₁S₂[t] (5.4)

and applying the usual pseudosteady-state approximation for the complex formation (ES₁ S₂[t]), we find:

$${ES}_{1} {S}_{2} [ t ] = \frac{{E}_{T} [ t ]}{1 + {Keq}_{1} {S}_{1} [ t ] + {Keq}_{2} {S}_{2} [ t ] + \frac{( {Keq}_{1} {kf}_{2} + {Keq}_{2} {kf}_{1} )}{2 k + {kb}_{1} + {kb}_{2}}} {S}_{1} [ t ] {S}_{2} [ t ] \qquad ( 5.5 )$$

and, on substitution:

$$\begin{matrix}\frac{ⅆ P [ t ]}{ⅆ t} = \frac{{kE}_{T} [ t ] {S}_{1} [ t ] {S}_{2} [ t ]}{{K}_{m} ( 1 + {Keq}_{1} {S}_{1} [ t ] + {Keq}_{2} {S}_{2} [ t ] ) + {S}_{1} [ t ] {S}_{2} [ t ]} & ( 5.6 ) \\ where , {K}_{m} = \frac{( k + {kb}_{1} ) + ( k + {kb}_{2} )}{{Keq}_{2} {kf}_{1} + {Keq}_{1} {kf}_{2}} & ( 5.7 )\end{matrix}$$

We can represent the variation in each variable as deviations from a control or steady-state concentration, as:

S₁[t]=S₁^{SS}+ΔS₁[t] (5.8)

S₂[t]=S₂^{SS}+ΔS₂[t] (5.9)

E_{T}[t]=E_{SS}+ΔE[t] (5.10)

P[t]=P_{SS}+ΔP[t] (5.11)

$$\begin{matrix}and , \frac{ⅆ P [ t ]}{ⅆ t} = \frac{ⅆ {P}_{ss}}{ⅆ t} + \frac{ⅆ Δ P [ t ]}{ⅆ t} & ( 5.12 ) \\ where , \frac{ⅆ {P}_{ss}}{ⅆ t} = \frac{{kE}_{ss} {S}_{1}^{ss} {S}_{2}^{ss}}{{K}_{m} ( 1 + {Keq}_{1} {S}_{1}^{ss} + {Keq}_{2} {S}_{2}^{ss} ) + {S}_{1}^{ss} {S}_{2}^{ss}} & ( 5.13 )\end{matrix}$$

Solving for the rate of change in the deviation of the product concentration (AP[t]), we get:

$$\frac{ⅆ Δ P [ t ]}{ⅆ t} = \frac{k ( {E}_{ss} + Δ E [ t ] ) ( {S}_{1}^{ss} + Δ {S}_{1} [ t ] ) ( {S}_{2}^{ss} + Δ {S}_{2} [ t ] )}{{K}_{m} \{ 1 + {Keq}_{1} ( {S}_{1}^{ss} + Δ {S}_{1} [ t ] ) + {Keq}_{2} ( {S}_{2}^{ss} + Δ {S}_{2} [ t ] ) \} + ( {S}_{1}^{ss} + Δ {S}_{1} [ t ] ) ( {S}_{2}^{ss} + Δ {S}_{2} [ t ] )} - \frac{{kE}_{ss} {S}_{1}^{ss} {S}_{2}^{ss}}{{K}_{m} ( 1 + {Keq}_{1} {S}_{1}^{ss} + {Keq}_{2} {S}_{2}^{ss} ) + {S}_{1}^{ss} {S}_{2}^{ss}} \qquad ( 5.14 )$$

Taking the Laplace transform, neglecting the deviations from the control values in the denominator of equation 5.14, and neglect the second order deviation terms, we get the transfer function:

$$\begin{matrix}P [ s ] = \frac{{G}_{1}}{s} Δ E [ s ] + \frac{{G}_{2}}{s} Δ {S}_{1} [ s ] + \frac{{G}_{3}}{s} Δ {S}_{2} [ s ] & ( 5.15 ) \\ where , & \\ {G}_{1} = \frac{{kS}_{1}^{ss} {S}_{2}^{ss}}{{K}_{m} ( 1 + {Keq}_{1} {S}_{1}^{ss} + {Keq}_{2} {S}_{2}^{ss} ) + {S}_{1}^{ss} {S}_{2}^{ss}} & ( 5.16 ) \\ {G}_{2} = \frac{{kE}_{ss} {S}_{2}^{ss}}{{K}_{m} ( 1 + {Keq}_{1} {S}_{1}^{ss} + {Keq}_{2} {S}_{2}^{ss} ) + {S}_{1}^{ss} {S}_{2}^{ss}} & ( 5.17 ) \\ {G}_{3} = \frac{{kE}_{ss} {S}_{1}^{ss}}{{K}_{m} ( 1 + {Keq}_{1} {S}_{1}^{ss} + {Keq}_{2} {S}_{2}^{ss} ) + {S}_{1}^{ss} {S}_{2}^{ss}} & ( 5.18 )\end{matrix}$$

Taking the inverse Laplace transform of equation 5.15 yields the solution to the difference equation in the

time domain,

ΔP[t]=G₁ΔE[t]+G₂S₁[t]+G₃S₂[t] (5.19)

### Example 6

### Michaelis-Menton Enzyme with Allosteric Upregulation

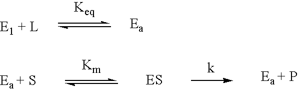

It is assumed that the binding of the regulatory ligand (L) is quick relative to the reaction catalyzed

by the activated enzyme, such that this can be represented by the equilibrium expression:

Eₐ[t]=K_{eq}E₁[t]L[t] (6.1)

In addition, it is noted that Ea[t] in this equilibrium expression is actually the concentration of the active enzyme plus the activated enzyme-substrate complex.

From Michaelis-Menton, we know that:

$$\frac{ⅆ P [ t ]}{ⅆ t} = \frac{{kE}_{a} [ t ] S [ t ]}{Km + S [ t ]} \qquad ( 6.2 )$$

But, the active enzyme concentration (Eₐ[t]) can be related to the total enzyme concentration (E[t]) from

the material balance:

E[t]=Eₐ[t]+Eᵢ[t] (6.3)

Therefore, combining equations 6.1, 6.2, and 6.3, the rate of product production can be represented as:

$$\frac{ⅆ P [ t ]}{ⅆ t} = \frac{{kK}_{eq} E [ t ] L [ t ] S [ t ]}{( 1 + {K}_{eq} L [ t ] ) ( Km + S [ t ] )} \qquad ( 6.4 )$$

The changes in each variable can be represented as deviations from their control or steady-state

concentrations:

E[t]=Eₛₛ+ΔE[t] (6.5)

S[t]=Sₛₛ+ΔS[t] (6.6)

L[t]=Lₛₛ+ΔL[t] (6.7)

P[t]=Pₛₛ+ΔP[t] (6.8)

$$\begin{matrix}and , \frac{ⅆ P [ t ]}{ⅆ t} = \frac{ⅆ {P}_{ss}}{ⅆ t} + \frac{ⅆ Δ P [ t ]}{ⅆ t} & ( 6.9 ) \\ where , \frac{ⅆ {P}_{ss}}{ⅆ t} = \frac{{kK}_{eq} {E}_{ss} {L}_{ss} {S}_{ss}}{( 1 + {K}_{eq} {L}_{ss} ) ( Km + {S}_{ss} )} & ( 6.10 )\end{matrix}$$

On substitution into equation 6.4, and solving for the rate of change of the product deviation (ΔP′[t]) we get:

$$\frac{ⅆ Δ P [ t ]}{ⅆ t} = \frac{k {K}_{eq} ( {E}_{SS} + Δ E [ t ] ) ( {L}_{SS} + Δ L [ t ] ) ( {S}_{SS} + Δ S [ t ] )}{\{ 1 + {K}_{eq} ( {L}_{SS} + Δ L [ t ] ) \} \{ Km + ( {S}_{ss} + Δ S [ t ] ) \}} - \frac{{kK}_{eq} {E}_{ss} {L}_{ss} {S}_{ss}}{( 1 + {K}_{eq} {L}_{ss} ) ( Km + {S}_{ss} )} \qquad \text{(6.11)}$$

However, we can assume that the deviations from steady-state in the denominator of the first term are negligible, to a first approximation. Furthermore, we can neglect the second order deviation terms, such that equation 6.11 reduces to the approximation:

$$\frac{ⅆ Δ P [ t ]}{ⅆ t} \approx \frac{{kK}_{eq} \{ {L}_{ss} {S}_{ss} Δ E [ t ] + {E}_{ss} {S}_{ss} Δ L [ t ] + {E}_{ss} {L}_{ss} Δ S [ t ] \}}{( 1 + {K}_{eq} {L}_{ss} ) ( Km + {S}_{ss} )} \qquad \text{(6.12)}$$

Taking the Laplace transform, we can find the transfer functions for each deviation variable:

$$\begin{matrix}Δ P [ s ] = \frac{{G}_{1}}{s} Δ E [ s ] + \frac{{G}_{2}}{s} Δ L [ s ] + \frac{{G}_{3}}{s} Δ S [ s ] \\ \text{where,} & \text{(6.13)} \\ {G}_{1} = \frac{{kK}_{eq} {L}_{ss} {S}_{ss}}{( 1 + {K}_{eq} {L}_{ss} ) ( Km + {S}_{ss} )} & \text{(6.14)} \\ {G}_{2} = \frac{{kK}_{eq} {E}_{ss} {S}_{ss}}{( 1 + {K}_{eq} {L}_{ss} ) ( Km + {S}_{ss} )} & \text{(6.15)} \\ {G}_{3} = \frac{{kK}_{eq} {E}_{ss} {L}_{ss}}{( 1 + {K}_{eq} {L}_{ss} ) ( Km + {S}_{ss} )} & \text{(6.16)}\end{matrix}$$

Taking the inverse Laplace transform, we can then find the solution to the difference equation in the time domain:

ΔP[t]=G₁ΔE[t]+G₂ΔL[t]+G₃ΔS[t] (6.17)

### Example 7

### Michaelis-Menton Enzyme with Allosteric Repression

$${E}_{a} + L {⇌}^{ {K}_{eq }} {E}_{i}$$

$${E}_{a} + S {⇌}^{ {K}_{m }} ES {\to }^{ k } {E}_{a} + P$$

It is assumed that the binding of the regulatory ligand (L) is quick relative to the reaction catalyzed

by the activated enzyme, such that this can be represented by the equilibrium expression:

Eₐ[t]=K_{eq}Eᵢ[t]L[t] (6.1)

In addition, it is noted that Ea[t] in this equilibrium expression is actually the concentration of the active enzyme plus the activated enzyme-substrate complex.

From Michaelis-Menton, we know that:

$$\frac{ⅆ P [ t ]}{ⅆ t} = \frac{k {E}_{a} [ t ] S [ t ]}{Km + S [ t ]} \qquad \text{(6.2)}$$

But, the active enzyme concentration (Eₐ[t]) can be related to the total enzyme concentration (E[t]) from the material balance:

E[t]=Eₐ[t]+Eᵢ[t] (6.3)

Therefore, combining equations 6.1, 6.2, and 6.3, the rate of product production can be represented as:

$$\frac{ⅆ P [ t ]}{ⅆ t} = \frac{k ( 1 - \frac{{K}_{eq} L [ t ]}{1 + {K}_{eq} L [ t ]} ) E [ t ] L [ t ] S [ t ]}{Km + S [ t ]} \qquad \text{(6.4)}$$

The changes in each variable can be represented as deviations from their control or steady-state concentrations:

E[t]=Eₛₛ+ΔE[t] (6.5)

S[t]=Sₛₛ+ΔS[t] (6.6)

L[t]=Lₛₛ+ΔL[t] (6.7)

P[t]=Pₛₛ+ΔP[t] (6.8)

$$\text{and,}$$

$$\begin{matrix}\frac{ⅆ P [ t ]}{ⅆ t} = \frac{ⅆ {P}_{ss}}{ⅆ t} + \frac{ⅆ Δ P [ t ]}{ⅆ t} \\ \text{where,} & \text{(6.9)} \\ \frac{ⅆ {P}_{ss}}{ⅆ t} = \frac{{kE}_{ss} {S}_{ss}}{( 1 + {K}_{eq} {L}_{ss} ) ( Km + {S}_{ss} )} & \text{(6.10)}\end{matrix}$$

On substitution into equation 6.4, and solving for the rate of change of the product deviation (ΔP′[t]) we get:

$$\frac{ⅆ Δ P [ t ]}{ⅆ t} = \frac{k ( 1 - \frac{{K}_{eq} ( {L}_{ss} + Δ L [ t ] )}{1 + {K}_{eq} ( {L}_{ss} + Δ L [ t ] )} ) ( {E}_{ss} + Δ E [ t ] ) ( {S}_{ss} + Δ S [ t ] )}{Km + ( {S}_{ss} + Δ S [ t ] )} - \frac{{kE}_{ss} {S}_{ss}}{( 1 + {K}_{eq} {L}_{ss} ) ( Km + {S}_{ss} )} \qquad \text{(6.11)}$$

However, we can assume that the deviations from steady-state in the denominators of the first term are negligible, to a first approximation.

$$\frac{ⅆ Δ P [ t ]}{ⅆ t} \approx - \frac{k ( 1 - {K}_{eq} Δ L [ t ] ) ( {E}_{ss} + Δ E [ t ] ) ( {S}_{ss} + Δ S [ t ] )}{( 1 + {K}_{eq} {L}_{ss} ) ( Km + {S}_{ss} )} \qquad \text{(6.12)}$$

Expanding this expression, eliminating the second and third order deviation terms, and taking the Laplace transform, we can find the transfer functions for each deviation variable:

$$\begin{matrix}Δ P [ s ] = \frac{{G}_{1}}{s} Δ E [ s ] + \frac{{G}_{2}}{s} Δ L [ s ] + \frac{{G}_{3}}{s} Δ S [ s ] \\ \text{where,} & \text{(6.13)} \\ {G}_{1} = \frac{k {S}_{ss}}{( 1 + {K}_{eq} {L}_{ss} ) ( Km + {S}_{ss} )} & \text{(6.14)} \\ {G}_{2} = \frac{{kK}_{eq} {E}_{ss} {S}_{ss}}{( 1 + {K}_{eq} {L}_{ss} ) ( Km + {S}_{ss} )} & \text{(6.15)} \\ {G}_{3} = \frac{{kE}_{ss}}{( 1 + {K}_{eq} {L}_{ss} ) ( Km + {S}_{ss} )} & \text{(6.16)}\end{matrix}$$

Taking the inverse Laplace transform, we can then find the solution to the difference equation in the time domain:

ΔP[t]=G₁ΔE[t]+G₂ΔL[t]+G₃ΔS[t] (6.17)

We claim:

1. A method comprising:
a) providing a plurality of unit operations that represent all or a subset of all actions that can be done on a set of system components;
b) providing a first hypothetical mathematical model comprising a subset of said unit operations;
c) applying a first artificial intelligence (Al) algorithm to said first hypothetical mathematical model to produce a second hypothetical mathematical model;
d) using a fitness function to filter said second hypothetical model to generate at least a third hypothetical mathematical model.

2. A method according to claim 1 wherein said fitness function comprises a goodness of fit comparison with at least a first set of empirical data.

3. A method according to claim 1 and 2 wherein said fitness function comprises at least one weighting factor to adjust a least a first adjustable parameter of said data.

4 A method according to claim 1, 2 or 3 wherein said method further comprises iterating steps c) and d).

5. A method according to claims 1, 2, 3 or 4 wherein said Al algorithm is a genetic algorithm.

6. A method according to claims 1, 2, 3, 4 or 5 wherein said fitness function utilizes a comparison to a first set of empirical data.

7. A method according to claim 6 wherein said first set comprises a set of differential display data points.

8. A method according to claim 7 wherein said differential data points comprise differential gene expression data points.

9. A method according to claim 8 wherein said differential gene expression data points are generated from cancerous tissue compared to normal tissue.

10. A method according to claim 9 wherein said cancerous tissue is selected from the group of cancerous tissues consisting of breast, prostate, lung, brain, ovarian, pancreas, liver, kidney, bladder and heart.

11. A method according to claim 7 wherein said differential display data points comprise differential weather pattern points.

12. A method according to claim 7 wherein said differential display data points comprise differential traffic pattern data points.

13. A method according to claim 7 wherein said differential display data points comprises differential financial market data points.

14. A method to generate a mathematical model of a biological system comprising:
a) providing a plurality of first order pseudogene unit operations that define a set of biochemical system parameters;
b) generating a first set of first order pseudochromosomes from said pseudogenes;
b) applying a genetic algorithm with a fitness function to said set of first order pseudochromosomes to produce a second set of second order pseudochromosomes;
c) comparing said second set of second order pseudochromosomes to at least a first set of empirical data;
d) optionally reiterating steps b) and c) to generate a global optimum solution comprising said mathematical model.

15. A method according to claim 14 wherein said first set of empirical data comprises a set of differential data points.

16. A method according to claim 15 wherein said differential data points comprise differential gene expression data points.

17. A method according to claim 15 wherein said differential gene expression data points are generated from cancerous tissue compared to normal tissue.

18. A method comprising:
a) providing a plurality of unit operations that define a set of system parameters;
b) providing a first hypothetical mathematical model comprising a subset of said unit operations;
c) applying a first artificial intelligence (Al) algorithm to said plurality to produce a second hypothetical mathematical model;
c) comparing said second hypothetical model to at least a first set of empirical data to define at least a first difference between said first hypothetical model and said data;
d) altering said first algorithm to adjust for said first difference to generate a second Al algorithm;
e) applying said second Al algorithm to said second hypothetical model to produce a third hypothetical model; and
f) comparing said third hypothetical model to said first set of data.

19. A computer readable memory to direct a computer to function in a specified manner, comprising:
a) a unit operations module to receive and store unit operations and generate at least a first hypothetical mathematical model;
b) an analysis module to apply an artificial intelligence algorithm;
c) a comparison module to compare hypothetical models to at least a first set of empirical data.
